(19) United States
(12) Patent Application Publication
(10) Pub. No.: US 2024/0399360 A1
(43) Pub. Date: Dec. 5, 2024

(54) DEVICES AND METHODS FOR CREATING TRANSCELLULAR PORES

(71) Applicants: Georgia Tech Research Corporation, Atlanta, GA (US); Columbia University, New York, NY (US); The Jackson Laboratory, Bar Harbor, ME (US); Imperial College London, London (GB); Duke University, Durham, NC (US)

(72) Inventors: Ross Ethier, Atlanta, GA (US); Simon John, New York, NY (US); Krishnakumar Kizhatil, Bar Harbor, ME (US); Darryl Overby, London (GB); Seyed Mohammad Siadat, Atlanta, GA (US); W. Daniel Stamer, Durham, NC (US); Joseph van Batenburg-Sherwood, London (GB)

(21) Appl. No.: 18/680,164

(22) Filed: May 31, 2024

Related U.S. Application Data
(60) Provisional application No. 63/505,804, filed on Jun. 2, 2023.

(51) Int. Cl. B01L 3/00; C12N 5/00; C12N 5/071

(52) U.S. Cl. CPC B01L 3/5027; C12N 5/0068; C12N 5/069; B01L 2300/0819; B01L 2300/16; C12N 2503/02

(57) ABSTRACT

Disclosed herein is a biochip with a plurality of endothelial cells (e.g., Schlemm's canal cells) having a plurality of pores (e.g., transcellular pores) and methods of making and using the same.

### CROSS REFERENCE TO RELATED APPLICATIONS

This application claims the benefit of priority to U.S. Provisional Application No. 63/505,804, filed Jun. 2, 2023, which is incorporated by reference herein in its entirety.

### STATEMENT REGARDING FEDERALLY SPONSORED RESEARCH

This invention was made with government support under Grant No. R21EY033142 awarded by the National Institutes of Health. The government has certain rights in the invention.

### BACKGROUND

Glaucoma is the leading global cause of irreversible blindness. Although glaucoma can occur at any level of intraocular pressure (IOP), elevated IOP is a primary risk factor, and is the only treatable risk factor. IOP is largely determined by the resistance to aqueous humor flow generated by the tissues of the conventional outflow pathway, specifically the trabecular meshwork and the inner wall of Schlemm's canal (SC). In particular, inner wall SC cells play a key role in homeostatic control mechanisms that maintain IOP within a target range.

All conventional outflow of aqueous humor crosses inner wall SC cells through micron-sized pores. There are two pore types: transcellular pores (intracellular or I-pores) and paracellular pores (border or B-pores). These pores have different sizes and dependencies on IOP, but both may facilitate aqueous humor drainage. Mechanical stretch triggers pore formation, and SC endothelium is exposed to significant stretch due to the pressure drop across the inner wall, most often during giant vacuole formation. It is important to note that pores are not tears or ruptures in the cell, but membrane-delineated structures that are actively formed by SC cells.

The discovery of SC-active agents that exploit and enhance native processes controlling SC hydraulic conductivity offers strategies for IOP control, yet such discovery is impeded by poor assays. Thus, there is a need for improved models of Schlemm's canal and assays made therefrom. These needs and others are at least partially satisfied by the present disclosure.

### SUMMARY

In an aspect, provided herein is a biochip, including: a substrate; a mechanical stressor; and a plurality of endothelial cells seeded on top of the mechanical stressor; wherein the plurality of endothelial cells can include a plurality of pores.

In another aspect, provided is a drug-screening assay including any of the disclosed biochips.

In yet another aspect, provided is a method of making an endothelial barrier model, the method including: a) providing a substrate including a mechanical stressor; b) seeding a plurality of endothelial cells on top of the mechanical stressor; and c) inducing transcellular pore formation in the plurality of endothelial cells using the mechanical stressor.

In yet another aspect, provided is a method of making a Schlemm's canal model, the method including: a) providing a substrate including a mechanical stressor; b) seeding a plurality of Schlemm's canal (SC) cells on top of the mechanical stressor; and c) inducing transcellular pore formation in the plurality of SC cells using the mechanical stressor.

In yet another aspect, provided is a method including: coating a glass substrate with biotinylated gelatin cross-linked with microbial transglutaminase; seeding the coated glass substrate with carboxyl ferromagnetic particles and, subsequently, primary human Schlemm's canal (SC) cells; adding a first tracer to the coated and seeded glass substrate; washing the first tracer from the coated and seeded glass substrate after a first time period; adding a second tracer to the coated and seeded glass substrate; applying a magnetic force to the coated and seeded glass substrate to create local cellular stretch in the primary human SC cells due to the carboxyl ferromagnetic particles; and imaging the primary human SC cells to identify tracer signals.

In yet another aspect, provided is a method of screening agents that modulate transcellular pore formation, the method including: i) providing an endothelial barrier model prepared by any of the disclosed methods; ii) exposing the endothelial barrier model to a therapeutic agent; and iii) determining the presence of transcellular pores in the plurality of endothelial cells as compared to an endothelial barrier model not exposed to the therapeutic agent.

In yet another aspect, provided is a method of screening agents that modulate transcellular pore formation, the method including: i) providing a Schlemm's canal model prepared by any of the disclosed methods; ii) exposing the Schlemm's canal model to a therapeutic agent; and iii) determining the presence of transcellular pores in the plurality of SC cells as compared to a Schlemm's canal model not exposed to the therapeutic agent.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIGS. 1A-1E depict pore formation in SC inner wall cells. FIG. 1A shows a diagram of an anatomical overview of aqueous humor (AH) outflow pathway. AH (blue arrows) is produced by the ciliary body, flows into the anterior chamber, and exits the anterior chamber primarily through the trabecular meshwork (TM) and SC. The magnified inset shows AH flows through consecutive regions of TM: uveal meshwork (UM), corneoscleral meshwork (CM), and juxtacanalicular tissue (JCT). AH then crosses SC inner wall cells through transcellular or paracellular pores. Basement membrane (BM) and extracellular matrix components are shown in grey. FIG. 1B shows a representative transmission electron micrograph showing significant deformation experienced by SC inner wall endothelial cells during giant vacuole (GV) formation. GVs are outpouchings of the endothelial cells lining SC, protruding into the canal's lumen and creating a fluid-filled space between the cell and the underlying BM. n: Nucleus; *: Opening in BM. FIG. 1C shows a representative scanning electron micrograph of the SC inner wall as observed from inside the canal. GVs are often associated with development of transcellular pores, which create transcellular routes for drainage of AH from anterior chamber of the eye into the SC lumen. Note the bulging structures (GVs and nuclei) and transcellular pores (arrowheads) passing through GVs. FIG. 1D is a schematic illustrating transcellular pore formation in a SC cell in vivo exposed to significant stretch due to the pressure drop across the inner wall, during GV formation.

FIG. 1E is a schematic illustrating transcellular pore formation in a SC cell induced by a particle in vitro. In the in vitro setup, phSC cells are seeded over a gelatin coated glass substrate and ˜5 μm carboxyl-coated, ferromagnetic particles to create local cellular deformation. The particles are about the same size as the GVs in vivo. It is hypothesized that as cells spread over these particles, they become thinner which ultimately gives rise to transcellular pores. P: particle; green: gelatin substrate underlying cells in culture.

FIGS. 2A-2F depict scanning electron micrographs demonstrating particle-induced cell deformation and transcellular pore formation. FIGS. 2A-2B are representative micrographs showing particle induced cell deformation. Left panels show low-magnification images of primary human Schlemm's canal (phSC) cells overlying particles, while right panels show higher-magnification views of the insets (black boxes), highlighting cell deformation at the particle locations. FIGS. 2C-2F show SEM micrographs demonstrating various aspects of particle-induced transcellular pores. FIG. 2A shows stretching of a phSC cell (suggested by black dashed arrows) associated with a particle near the cell's border (white arrowheads). Although this particle did not induce the formation of a transcellular pore, noticeable cellular deformation around the particle is evident. FIG. 2B shows a transcellular pore (black arrow) associated with a particle. Note the artifactual cracks (black arrowheads) with irregular, jagged edges near the particle. FIG. 2C shows a transcellular pore formed directly above a particle, with several noticeable cracks in close proximity. FIG. 2D shows a transcellular pore formed to the side of the particle, situated near the cell border. FIG. 2E shows two transcellular pores formed on the side of a particle, elongated in the same direction as the cell stretched. FIG. 2F shows multiple transcellular pores formed above a particle, resulting in a “toffee-like” structure. Black arrows indicate transcellular pores, black arrowheads indicate artifactual pores or cracks with jagged edges, white arrowheads indicate cell borders, and black dashed arrows indicate the apparent direction of cell stretching.

FIGS. 3A-3G depict cell deformation via particles and transcellular pore detection without SEM imaging. FIG. 3A shows a schematic illustrating the fluorescent assay (side view) for rapid transcellular pore detection. phSC cells are grown on biotinylated gelatin substrates, and the presence of transcellular pores is revealed using a label (fluorescently labeled streptavidin). FIG. 3B is a schematic illustrating that after imaging cells via fluorescent microscopy, transcellular pores are detectable as bright spots in the middle of dark islands that represent cells' footprint. FIG. 3C shows a step-by-step schematic of the mechanobiological assay tailored for transcellular pore induction and detection. First, glass substrates were coated with biotinylated gelatin. Particles were then randomly seeded on the biotinylated gelatin for 10 minutes. phSC cells were then seeded over particles and cultured for 2-7 hours to fully spread. Finally, the pore label was added to media for 5 minutes to detect transcellular pores. Cells were then fixed and imaged. The particle is shown in grey or dotted circles. FIG. 3D presents confocal images showing significant cellular deformation localized at the particle site. The leftmost panel and center panel show top view, maximum intensity projections of a phSC cell labeled for cell cytoplasm and nucleus (red). The cell is spread above a particle (white arrow). The pore label (shown in green) is bound to the biotinylated gelatin substrate outside of the cell footprint. The rightmost panel shows a cross-sectional side view of the cell at the location of the particle (along the dashed line in the center panel) highlighting cell thinning above the particle (arrow). FIG. 3E shows a graph estimating cell thickness from confocal images. Cells were significantly thinner above the particles (**** p<0.0001). FIG. 3F shows a graph estimating cell thickness at nuclear and non-nuclear regions. FIG. 3G presents confocal images showing both the cellular deformation and successful transcellular pore detection at the microparticle region (left and middle panels: top view, maximum intensity projections; right panel: cross-sectional side view). A phSC cell is spread above a particle (white arrow). The pore label is bound to the biotinylated substrate outside of the cell footprint and at the border of neighboring cells due to lack of tight intercellular junctions. The pore label is also bound to the substrate at the location of the particle indicating transcellular pore formation (highlighted by the arrow). White arrow heads in panels d and g point to intracellular vesicles (see FIGS. 9A-9B).

FIGS. 4A-4F depict high content fluorescence detection of transcellular pores. FIG. 4A shows schematics of the assay. phSC cells were cultured over particles on a biotinylated gelatin substrate in 16-well chambered slides to allow for high-content detection of transcellular pores. Initially, cells were exposed to a first pore label (green) to detect any transcellular pores resulting from particle interaction alone. Subsequently, cells were treated to induce further pore formation or left untreated as controls to assess pore dynamics. Pores induced by these treatments or new pores emerging in control conditions at particle locations were detected using a second pore label (magenta). FIG. 4B shows a transient pore (i.e., a pore detected only by the first pore label, suggesting it had closed by the time of the second labeling). FIG. 4C shows an emergent pore (i.e., a pore detected only by the second pore label, suggesting it formed after the initial labeling). FIG. 4D shows a persistent pore (i.e., a pore detected by both the first and second pore labels, suggesting continued opening). Note the opening in the actin network at the pore location. FIG. 4E shows a non-responsive particle (i.e., a particle without any detectable pores). FIG. 4F shows a non-particle-associated pore: A pore detected by the fluorescent assay but not associated with any visible particle. Insets in FIGS. 4B-4E provide 2× magnified views at the location of particles. The first column in FIGS. 4B-4F show DAPI-stained nuclei (blue) merged with a brightfield image, where particles appear as black dots (white arrowheads) and are highlighted by dashed circles in other images. Actin network (red), first pore label (green), and second pore label (magenta) are shown in the second, third, and fourth columns of FIGS. 4B-4F, respectively.

FIGS. 5A-5H depict graphs quantifying particle-induced pores and emergent pores in normal and glaucoma cells. FIG. 5A shows a graph of particle-induced transcellular pores detected by the first pore label (“Particle-induced Pores”) in normal (blue bars, circular data points) and glaucomatous (orange bars, square data points) cell strains, presented as the percentage of pores per particle. The color and shape of data points for each cell strain remain consistent throughout the entire figure. FIG. 5B is a graph of pooled data from FIG. 5A comparing particle-induced transcellular pores in normal and glaucomatous cells strains. FIG. 5C shows a graph of the percentage of particle-induced pores in each cell strain that were detected by the first pore label, as shown in FIG. 5A, but were closed at the time of exposure to the second pore label (“Transient Pores”). FIG. 5D is a graph of pooled data from FIG. 5C, comparing transient pores in normal and glaucomatous cell strains. FIG. 5E shows a graph of the percentage of particles that induced new pores detected only by the second pore label (“Emergent Pores”). FIG. 5F shows a graph of pooled data from FIG. 5E, comparing emergent pores in normal and glaucomatous cell strains. FIGS. 5G-5H show graphs of the percentage of emergent pores detected only by the second pore label in normal (FIG. 5G) and glaucomatous (FIG. 5H) cell strains exposed to no force (NF; control), low force (LF), medium force (MF), or high force (HF) conditions. n: normal; g: glaucomatous. Sec FIGS. 11A-11G for magnetic force application and measurement. Data are presented as mean±standard deviation. Statistical significance is indicated with (ns), (*), (**), (***), and (****) corresponding to not significant, p<0.05, p<0.01, p<0.001, and p<0.0001, respectively, except in FIGS. 5A, 5C, and 5E.

FIGS. 6A-6E depict the impact of cell stiffness on pore formation. FIG. 6A shows a graph of the stiffness of five normal cell strains and three glaucomatous cell strains measured by atomic force microscopy (AFM). FIG. 6B shows the linear regression of cell stiffness and particle-induced pore incidence (R²=0.41). Dotted lines show 95% confidence intervals. FIG. 6C shows a graph of cell stiffness. Cell stiffness was decreased after Y-27632 treatment in a dose-dependent manner in four normal cell strains. FIG. 6D shows a graph of particle pore formation after Y-27632 treatment. Pore formation was increased after Y-27632 treatment in a dose-dependent manner in four normal cell strains. FIG. 6E is the linear regression of cell stiffness and particle-induced pore incidence after Y-27632 and SIP treatments in four normal cell strains (R²=0.74). Data are presented as mean±standard deviation. Statistical significance is indicated with (ns), (*), (**), (***), and (****) corresponding to not significant, p<0.05, p<0.01, p<0.001, and p<0.0001, respectively.

FIGS. 7A-7B depict a proposed model of intracellular pore formation in phSC cells. FIG. 7A is an illustrated schematic of the model of transcellular pore formation in normal cells. In a normal phSC cell (top), GV formation results in substantial cell thinning (middle), eventually causing cell membranes to come into close proximity and induce pore formation (bottom). FIG. 7B is an illustrated model showing the lack of transcellular pore formation in glaucoma cells. In a stiff glaucomatous cell (top), GV formation is unable to sufficiently stretch and thin the cell (middle), preventing the cell membranes from coming close enough to facilitate pore formation (bottom). Cytoskeleton filaments, such as actin filaments, are depicted in red, cell nuclei in dark blue, AH in light blue, and both the extracellular matrix and BM in grey.

FIGS. 8A-8B depict that transcellular pores are induced by 2 μm particles seeded under phSC cells. FIG. 8A is representative SEM micrographs demonstrating particle-induced transcellular pore formation. The left image shows a low-magnification view of a phSC cell, while the right image shows a higher-magnification view of the inset (black box), highlighting the particle location and transcellular pore (white arrow). FIG. 8B shows brightfield and fluorescent images showing transcellular pores (white arrows) at the location of 2 μm particles. Pores were detected by the fluorescent assay (described in FIG. 4A) and without any treatment between the first and second pore labels. The left image shows DAPI-stained nuclei (blue) merged with a brightfield image in which particles are seen as black dots. The particles below cells are shown by white circles in other images. Actin network (red), first pore label (green), and second pore label (magenta) are shown in second, third, and fourth columns, respectively.

FIGS. 9A-9B show confocal images of cells seeded above a particle or without any particles. In both FIGS. 9A-9B, left and middle panels show top views, maximum intensity projections of a phSC cell labeled for cell cytoplasm and nucleus (red), while right and lower panels show cross-sectional side views of the cells at the location of intracellular vesicles (white arrowheads), along the A-A and B—B dashed lines in middle panel. Note that pore label (shown in green) is bound to the biotinylated gelatin substrate outside of the cell footprint, but also slightly reached under the cell at the cell boundaries (white arrows). The cell shown in FIG. 9A is the same cell shown in FIG. 3G.

FIG. 10 depicts a graph demonstrating weak association between pore formation and donor age in phSC cells via linear regression analysis. Linear regression analysis demonstrates a relationship between particle-induced pore incidence and the age of phSC cell donors (R²=0.19, p<0.0001). It's noteworthy that three cell strains derived from older donors had a prior history of glaucoma. Dotted lines represent 95% confidence intervals. Data are shown as mean±standard deviation.

FIGS. 11A-11G show the design and functionality of custom magnetic actuator. FIG. 11A shows a schematic of a 3D computer-aided design that illustrates the magnetic actuator, comprising a lower plate holder and an upper magnet holder. The upper magnet holder can slide down (indicated by red dashed arrows) to position the magnets at a controlled distance from the particles within the 16-well chambered slide. The device is engineered to exert magnetic force exclusively on the particles in the eight middle wells, while the remaining eight wells serve as control wells without magnets. FIG. 11B shows a schematic of various views of the upper magnet holder. Small spacers on the bottom surface of the upper magnet holder regulate the distance between the magnets ( 3/16″ diameter×½″ thick, D38-N52, K&J Magnetics) and the particles, while eight slots on the top surface of the upper magnet holder accommodate supporting magnets (¼″ diameter×½″ thick, D48-N52, K&J Magnetics) securing the D38-N52 magnets in position. FIG. 11C shows final configurations with magnets outside the wells and FIG. 11D shows final configurations with magnets inside the wells. FIGS. 11E-11F show the experimental setup for measuring the magnetic force exerted on each particle. Magnets are placed in a well-plate on a scale, zeroed (FIG. 11E). Using a high precision Z-axis stage, a coverslip is positioned slightly above the magnets without direct contact. A known quantity of particles is then pipetted above the magnets, and the scale is read (FIG. 11F) to calculate the axial force applied on the particles (see Methods). By incrementally adjusting the distance between magnets and particles using the Z-axis stage, the magnetic force applied on particles at different distances from the magnet surface is measured. FIG. 11G shows a graph of average measured applied force on each particle versus the distance from the magnets (x-axis error bars are ±20 μm). Three types of magnets were tested, with D28-N52 magnets (⅛″ diameter×½″ thick, K&J Magnetics) exhibiting the highest force at shorter distances. However, due to their smaller diameter, they limit the number of cells and particles that can be precisely examined under the magnet. Consequently, only D38-N52 magnets were utilized in experiments requiring magnetic force for cell stretching.

FIGS. 12A-12B display the impact of Y-27632 on cell area. FIG. 12A shows a graph of the area of cells treated with Y-27632. Y-27632 treatments showed no significant effect on the cell area. Analysis via 2-way ANOVA yielded the following P values: interaction=0.8, cell strain factor <0.0001, Y-27632 factor=0.4. FIG. 12B shows fluorescent images depicting the actin network (red) and cell nuclei (blue) in nSC74, nSC75, nSC82, and nSC89 cell strains following Y-27632 treatments.

FIGS. 13A-13B show graphs depicting the impact of SIP on cell stiffness and pore formation. FIG. 13A shows a graph of cell stiffness in cells treated with and without SIP. FIG. 13B shows a graph of pore formation in cells treated with and without SIP. SIP treatments resulted in a significant increase in cell stiffness (FIG. 13A) and a decrease in pore formation (FIG. 13B) in nSC75 and nSC89. However, SIP treatment did not affect cell stiffness or pore formation in nSC74. Data are shown as mean±standard deviation.

FIGS. 14A-14B depict that transcellular pores were not detected when particles were placed on top of phSC cells. FIG. 14A shows an illustration of the assay setup where phSC cells were cultured over biotinylated gelatin substrate in 16-well chambered slides, allowing for high-content detection of transcellular pores. Once cells were fully spread, particles were randomly positioned on top of the cells. A first pore label (green) was applied to detect any transcellular pores formed solely due to particles. Subsequently, particles were subjected to a magnetic field to induce local apical-to-basal cellular deformation, thereby promoting pore formation. Magnet-induced pores at the particle locations were identified using a second pore label (magenta). FIG. 14B shows representative micrographs from the assay in FIG. 14A. The top left image displays a brightfield image with particles represented as black dots, merged with fluorescent images of phSC cells exhibiting DAPI-stained nuclei (blue), the first pore label (green), and the second pore label (magenta). Other subpanels depict nuclei merged with the actin network (red), the first pore label, or the second pore label. No transcellular pore was detected at the particle locations. Treatment with 10 μM Y-27632 instead of applying a magnetic field also failed to induce transcellular pores.

FIGS. 15A-15D depict high resolution confocal images of particles placed on top of phSC cells. When particles were placed atop phSC cells, cell deformation was not observed, and transcellular pores were not detected. FIG. 15A shows the top view of a micrograph with the maximum intensity projections of a phSC cell labeled for cell cytoplasm (red) and nucleus (bluc). The cell is spread on biotinylated gelatin substrate while a particle (white arrow) is positioned atop the cell. FIG. 15B shows a micrograph of the pore label (green) bound to the biotinylated gelatin substrate outside of the cell footprint. FIG. 15C is a cross-sectional side view of the cell at the location of the particle (along the dashed line A-A in FIG. 15B), illustrating the particle's position on top of the cell. FIG. 15D is a micrograph similar to FIG. 15C but without showing the particle, highlighting the absence of cell deformation. The absence of pore label at the particle location indicates that no transcellular pore was formed.

FIGS. 16A-16C show micrographs revealing the induction of transcellular pores in HUVECs and HDMVECs by magnetic particles placed on cell surfaces. FIG. 16A shows low magnification brightfield and fluorescent images revealing transcellular pores in HUVECs where particles were positioned atop cells. Pores (white arrowheads) predominantly formed following the application of a magnetic field (473±11 pN per particle). Please note that the study did not quantify the number of pores before and after applying the magnetic field in these experiments. FIG. 16B shows low magnification and brightfield fluorescent images. No transcellular pores were observed when no particles were added atop HUVECs. FIG. 16C shows higher magnification images depicting transcellular pores in HDMVECs. Particles are visualized as black dots in brightfield images. The locations of particles are also indicated by white circles in FIG. 16C. DAPI-stained nuclei are depicted in blue, the actin network in red, the first pore label in green, and the second pore label in magenta.

FIGS. 17A-17D show representative fluorescent micrographs showing I-pores detected by the fluorescent assay colocalizing with magnetic particles under SC cells. FIG. 17A shows a brightfield image. Cell nuclei (blue) overlain by brightfield image showing particles (dark spots). Particles are outlined by white circles in FIGS. 17B-17D. FIG. 17B is a representative micrograph showing F-actin labeling (phalloidin; red). FIG. 17C shows a representative fluorescent micrograph displaying the first tracer (green). White arrow: Type I pore. FIG. 17D is a representative fluorescent micrograph displaying the second tracer (magenta). Yellow arrows: Type II pores. Note the disruption of the actin network in FIG. 17B at the position of Type II pores. Scale bars=20 μm.

FIG. 18 shows graphs of the number (left) and the area (right) of Type II pores minus Type I pores, normalized by the number of particles under subconfluent SC cells. Blue bars indicate normal SC cell strains (nSC89 and nSC82 shown with green and orange points, respectively), while red bars indicate glaucomatous SC cells (gSC57). Data shown as mean±standard deviations between wells. ª and b signify differences at p<0.05 by post hoc Tukey's HSD test.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g. desired antioxidant release rate or viscoelasticity. The specific level in terms of wt % in a composition required as an effective amount will depend upon a variety of factors including the amount and type of monomer, amount and type of polymer, e.g., acrylamide, amount of antioxidant, and desired release kinetics.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed drug delivery composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term “treatment” as used herein can include any treatment of a disease or disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

### Biochip Devices

In an aspect, provided is a biochip, including: a substrate; a mechanical stressor; and a plurality of endothelial cells seeded on top of the mechanical stressor; wherein the plurality of endothelial cells can include a plurality of pores. In some aspects, the plurality of pores can be formed by local cellular stretch over the mechanical stressor.

In some aspects, the substrate can include glass. In other aspects, the substrate can include a hydrogel (e.g., a PEG hydrogel, Matrigel, etc.). In yet other aspects, the substrate can include a tissue culture plastic. In yet other aspects, the substrate can include glass and/or a tissue culture plastic at least partially covered by a hydrogel. In some aspects, the substrate can include a biocompatible material that can be mechanically deformed.

In an aspect, provided is a biochip, including: a substrate; a mechanical stressor; and a plurality of endothelial cells seeded on top of the mechanical stressor.

In some aspects, the substrate can further include a coating. In some such aspects, the coating can include collagen, gelatin, biotin, fibronectin, and/or laminin. For example, in some aspects, the coating can include biotinylated, crosslinked gelatin. Additionally or alternatively, in some aspects, the coating can be configured to bind to a tracer at any regions of said coating not covered by the plurality of endothelial cells. Additionally or alternatively, in other aspects, the coating can induce or enhance local stress over the mechanical stressor.

In some aspects, the mechanical stressor can include a plurality of ferromagnetic particles. In some such aspects, the plurality of ferromagnetic particles can include carboxyl-coated ferromagnetic particles.

In some aspects, each of the plurality of ferromagnetic particles can have a diameter of at least about 1 μm (e.g., at least about 1.5 μm, at least about 2 μm, at least about 2.5 μm, at least about 3 μm, at least about 3.5 μm, at least about 4 μm, at least about 4.5 μm, at least about 5 μm, at least about 5.5 μm, at least about 6 μm, at least about 6.5 μm, at least about 7 μm, at least about 7.5 μm, at least about 8 μm, at least about 8.5 μm, at least about 9 μm, at least about 9.5 μm, at least about 10 μm). In some aspects, each of the plurality of ferromagnetic particles can have a diameter of up to about 10 μm (e.g., up to about 9.5 μm, up to about 9 μm, up to about 8.5 μm, up to about 8 μm, up to about 7.5 μm, up to about 7 μm, up to about 6.5 μm, up to about 6 μm, up to about 5.5 μm, up to about 5 μm, up to about 4.5 μm, up to about 4 μm, up to about 3.5 μm, up to about 3 μm, up to about 2.5 μm, up to about 2 μm, up to about 1.5 μm, up to about 1 μm). In some aspects, each of the plurality of ferromagnetic particles can have a diameter of about 1 μm, about 1.5 μm, about 2 μm, about 2.5 μm, about 3 μm, about 3.5 μm, about 4 μm, about 4.5 μm, about 5 μm, about 5.5 μm, about 6 μm, about 6.5 μm, about 7 μm, about 7.5 μm, about 8 μm, about 8.5 μm, about 9 μm, about 9.5 μm, or about 10 μm.

It is considered that each of the plurality of ferromagnetic particles can have a diameter ranging from any of the minimum values described above to any of the maximum values described above. For example, in some aspects, each of the plurality of ferromagnetic particles can have a diameter of from about 1 μm to about 10 μm (e.g., from about 1.5 μm to about 9.5 μm, from about 2 μm to about 9 μm, from about 2.5 μm to about 8.5 μm, from about 3 μm to about 8 μm, from about 3.5 μm to about 7.5 μm, from about 4 μm to about 7 μm, from about 4.5 μm to about 6.5 μm, from about 5 μm to about 6 μm, from about 1 μm to about 5.5 μm, from about 1.5 μm to about 5 μm, from about 2 μm to about 4.5 μm, from about 2.5 μm to about 4 μm, from about 3 μm to about 3.5 μm, from about 5.5 μm to about 10 μm, from about 6 μm to about 9.5 μm, from about 6.5 μm to about 9 μm, from about 7 μm to about 8.5 μm, from about 7.5 μm to about 8 μm).

In some aspects, each of the plurality of ferromagnetic particles can have approximately the same diameter (e.g., from about 1 μm to about 10 μm as described above). In other aspects, each of the plurality of ferromagnetic particles can have different diameters (e.g., from about 1 μm to about 10 μm as described above). In some aspects, the plurality of ferromagnetic particles can yield pores of approximately equal sizes. In other aspects, the plurality of ferromagnetic particles can yield pores of varying sizes.

In other aspects, the mechanical stressor can include a plurality of nonmagnetic nanoparticles and/or microparticles. In some such aspects, the mechanical stressor can include a plurality of ferromagnetic particles and a plurality of nonmagnetic nanoparticles and/or microparticles. In yet other aspects, the mechanical stressor can include a plurality of physical deformations (e.g., ridges, holes, bumps, etc.) in the substrate.

In some aspects, the mechanical stressor can be small enough to be covered by a cell and large enough to be detectable via microscopy.

In some aspects, the plurality of endothelial cells can include Schlemm's canal (SC) cells, vascular endothelial cells, microvascular endothelial cells, lymphatic endothelial cells, human umbilical vein endothelial cell (HUVECs), human dermal microvascular endothelial cells (HDMVECs), or any combination thereof. For example, in some aspects, the plurality of endothelial cells can include Schlemm's canal (SC) cells. In some such aspects, the SC cells can be healthy. In other such aspects, the SC cells can be diseased or abnormal. For example, in some aspects, the SC cells can be glaucomatous. In other aspects, the SC cells can be consistent with another disease or disorder associated with abnormal (i.e., abnormally high or abnormally low) intraocular pressure (IOP).

In other aspects, the plurality of endothelial cells can include human umbilical vein endothelial cells (HUVECs). In yet other aspects, the plurality of endothelial cells can include human dermal microvascular endothelial cells (HDMVECs).

In some aspects, the plurality of endothelial cells can be healthy. In other aspects, the plurality of endothelial cells can be diseased or abnormal. For example, in some such aspects, plurality of endothelial cells can be cancerous.

In some aspects, the plurality of endothelial cells can be obtained from the eye, kidney, brain, or another organ with endothelial tissues. In some aspects, the plurality of endothelial cells can be human.

In some aspects, the plurality of pores can include transcellular pores. As used herein, the term “transcellular pores,” “intracellular pores,” and “I-pores” refer to pores spanning in the basal-to-apical direction but not involving or touching upon a cell border. Additionally or alternatively, in other aspects, the plurality of pores can include paracellular pores. As used herein, the term “paracellular pores,” “intercellular pores,” or “B-pores” refers to paracellular pores, for example, pores intersecting the intercellular space between cells. Additionally or alternatively, in yet other aspects, the plurality of pores can include transcellular pores and paracellular pores.

In another aspect, provided is a drug-screening assay including any of the disclosed biochips.

### Methods

In an aspect, provided is a method of making an endothelial barrier model, the method including: a) providing a substrate including a mechanical stressor; b) seeding a plurality of endothelial barrier cells on top of the mechanical stressor; and c) inducing transcellular pore formation in the plurality of endothelial cells using the mechanical stressor. In some aspects, transcellular pore formation can be induced by local cellular stretch over the mechanical stressor.

In some aspects, the method can further include: d) exposing the plurality of endothelial cells to a tracer, wherein the tracer can bind to the substrate (e.g., the substrate or a coating on the substrate) at any regions of said substrate not covered by the plurality of endothelial cells; and e) detecting the tracer, thereby determining the presence of transcellular pores in the endothelial cells.

In some aspects, the tracer can include a fluorophore or fluorescent dye. In some such aspects, the tracer can include fluorescently labeled streptavidins (FL-SA).

In some aspects, the substrate can include glass. In other aspects, the substrate can include a hydrogel (e.g., a PEG hydrogel, Matrigel, etc.). In yet other aspects, the substrate can include glass at least partially covered by a hydrogel. In some aspects, the substrate can include a biocompatible material that can be mechanically deformed.

In some aspects, the substrate can further include a coating. In some such aspects, the coating can include collagen, gelatin, biotin, fibronectin, and/or laminin. For example, in some aspects, the coating can include biotinylated, crosslinked gelatin. Additionally or alternatively, in some aspects, the coating can be configured to bind to a tracer at any regions of said coating not covered by the plurality of endothelial cells. Additionally or alternatively, in other aspects, the coating can induce or enhance local stress over the mechanical stressor.

In some aspects, the mechanical stressor can include a plurality of ferromagnetic particles. In some such aspects, the plurality of ferromagnetic particles can include carboxyl-coated ferromagnetic particles.

In some aspects, each of the plurality of ferromagnetic particles can have a diameter of at least about 1 μm (e.g., at least about 1.5 μm, at least about 2 μm, at least about 2.5 μm, at least about 3 μm, at least about 3.5 μm, at least about 4 μm, at least about 4.5 μm, at least about 5 μm, at least about 5.5 μm, at least about 6 μm, at least about 6.5 μm, at least about 7 μm, at least about 7.5 μm, at least about 8 μm, at least about 8.5 μm, at least about 9 μm, at least about 9.5 μm, at least about 10 μm). In some aspects, each of the plurality of ferromagnetic particles can have a diameter of up to about 10 μm (e.g., up to about 9.5 μm, up to about 9 μm, up to about 8.5 μm, up to about 8 μm, up to about 7.5 μm, up to about 7 μm, up to about 6.5 μm, up to about 6 μm, up to about 5.5 μm, up to about 5 μm, up to about 4.5 μm, up to about 4 μm, up to about 3.5 μm, up to about 3 μm, up to about 2.5 μm, up to about 2 μm, up to about 1.5 μm, up to about 1 μm). In some aspects, each of the plurality of ferromagnetic particles can have a diameter of about 1 μm, about 1.5 μm, about 2 μm, about 2.5 μm, about 3 μm, about 3.5 μm, about 4 μm, about 4.5 μm, about 5 μm, about 5.5 μm, about 6 μm, about 6.5 μm, about 7 μm, about 7.5 μm, about 8 μm, about 8.5 μm, about 9 μm, about 9.5 μm, or about 10 μm.

It is considered that each of the plurality of ferromagnetic particles can have a diameter ranging from any of the minimum values described above to any of the maximum values described above. For example, in some aspects, each of the plurality of ferromagnetic particles can have a diameter of from about 1 μm to about 10 μm (e.g., from about 1.5 μm to about 9.5 μm, from about 2 μm to about 9 μm, from about 2.5 μm to about 8.5 μm, from about 3 μm to about 8 μm, from about 3.5 μm to about 7.5 μm, from about 4 μm to about 7 μm, from about 4.5 μm to about 6.5 μm, from about 5 μm to about 6 μm, from about 1 μm to about 5.5 μm, from about 1.5 μm to about 5 μm, from about 2 μm to about 4.5 μm, from about 2.5 μm to about 4 μm, from about 3 μm to about 3.5 μm, from about 5.5 μm to about 10 μm, from about 6 μm to about 9.5 μm, from about 6.5 μm to about 9 μm, from about 7 μm to about 8.5 μm, from about 7.5 μm to about 8 μm).

In some aspects, each of the plurality of ferromagnetic particles can have approximately the same diameter (e.g., from about 1 μm to about 10 μm as described above) to yield pores of approximately equal sizes. In other aspects, each of the plurality of ferromagnetic particles can have different diameters (e.g., from about 1 μm to about 10 μm as described above) to yield pores of varying sizes.

In some aspects, step c) can further include applying a magnetic force to the plurality of SC cells. In some aspects, the magnetic force can be at least about 400 N (e.g., at least about 450 N, at least about 500 N, at least about 550 N, at least about 600 N, at least about 650 N, at least about 700 N, at least about 750 N, at least about 800 N, at least about 850 N, at least about 900 N, at least about 950 N, at least about 1000 N, at least about 1050 N, at least about 1100 N, at least about 1150 N, at least about 1200 N, at least about 1250 N, at least about 1300 N, at least about 1350 N, at least about 1400 N, at least about 1450 N, at least about 1500 N). In some aspects, the magnetic force can be up to about 1500 N (e.g., up to about 1450 N, up to about 1400 N, up to about 1350 N, up to about 1300 N, up to about 1250 N, up to about 1200 N, up to about 1150 N, up to about 1100 N, up to about 1050 N, up to about 1000 N, up to about 950 N, up to about 900 N, up to about 850 N, up to about 800 N, up to about 750 N, up to about 700 N, up to about 650 N, up to about 600 N, up to about 550 N, up to about 500 N, up to about 450 N, up to about 400 N). In some aspects, the magnetic force can be about 400 N, about 450 N, about 500 N, about 550 N, about 600 N, about 650 N, about 700 N, about 750 N, about 800 N, about 850 N, about 900 N, about 950 N, about 1000 N, about 1050 N, about 1100 N, about 1150 N, about 1200 N, about 1250 N, about 1300 N, about 1350 N, about 1400 N, about 1450 N, or about 1500 N.

It is considered that the magnetic force can range from any of the minimum values described above to any of the maximum values described above. For example, in some aspects, the magnetic force can be from about 400 N to about 1500 N (e.g., from about 450 N to about 1450 N, from about 500 N to about 1400 N, from about 550 N to about 1350 N, from about 600 N to about 1300 N, from about 650 N to about 1250 N, from about 700 N to about 1200 N, from about 750 N to about 1150 N, from about 800 N to about 1100 N, from about 850 N to about 1050 N, from about 800 N to about 1000 N, from about 850 N to about 950 N, from about 400 N to about 900 N, from about 450 N to about 850 N, from about 500 N to about 800 N, from about 550 N to about 750 N, from about 600 N to about 700 N, form about 900 N to about 1500 N, from about 950 N to about 1450 N, from about 1000 N to about 1400 N, from about 1050 N to about 1350 N, from about 1100 N to about 1300 N, from about 1150 N to about 1250 N).

In some aspects, the magnetic force can be applied to the plurality of SC cells for at least about 1 minute (e.g., at least about 2 minutes, at least about 3 minutes, at least about 4 minutes, at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 60 minutes). In some aspects, the magnetic force can be applied to the plurality of SC cells for up to about 60 minutes (e.g., up to about 55 minutes, up to about 50 minutes, up to about 45 minutes, up to about 40 minutes, up to about 35 minutes, up to about 30 minutes, up to about 25 minutes, up to about 20 minutes, up to about 15 minutes, up to about 10 minutes, up to about 5 minutes, up to about 4 minutes, up to about 3 minutes, up to about 2 minutes, up to about 1 minute). In some aspects, the magnetic force can be applied to the plurality of SC cells for about 1 minute, about 2 minutes, about 3 minutes, about 4 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes.

It is considered that the magnetic force can be applied to the plurality of SC cells for a duration ranging from any of the minimum values described above to any of the maximum values described above. For example, in some aspects, the magnetic force can be applied to the plurality of SC cells for from about 1 minute to about 60 minutes (e.g., from about 2 minutes to about 55 minutes, from about 3 minutes to about 50 minutes, from about 4 minutes to about 45 minutes, from about 5 minutes to about 40 minutes, from about 10 minutes to about 35 minutes, from about 15 minutes to about 30 minutes, from about 20 minutes to about 25 minutes, from about 1 minute to about 25 minutes, from about 2 minutes to about 20 minutes, from about 3 minutes to about 15 minutes, from about 4 minutes to about 10 minutes, from about 20 minutes to about 60 minutes, from about 25 minutes to about 55 minutes, from about 30 minutes to about 50 minutes, from about 35 minutes to about 45 minutes).

In other aspects, the mechanical stressor can include a plurality of nonmagnetic nanoparticles and/or microparticles. In some such aspects, the mechanical stressor can include a plurality of ferromagnetic particles and a plurality of nonmagnetic nanoparticles and/or microparticles. In yet other aspects, the mechanical stressor can include a plurality of physical deformations (e.g., ridges, holes, bumps, etc.) in the substrate.

In some aspects, the plurality of endothelial cells can include Schlemm's canal (SC) cells, vascular endothelial cells, microvascular endothelial cells, lymphatic endothelial cells, or any combination thereof. For example, in some aspects, the plurality of endothelial cells can include Schlemm's canal (SC) cells. In some such aspects, the SC cells can be healthy. In other such aspects, the SC cells can be diseased or abnormal. For example, in some aspects, the SC cells can be glaucomatous. In other aspects, the SC cells can be consistent with another disease or disorder associated with abnormal (i.e., abnormally high or abnormally low) intraocular pressure (IOP).

In some aspects, the plurality of endothelial cells can be healthy. In other aspects, the plurality of endothelial cells can be diseased or abnormal. For example, in some such aspects, plurality of endothelial cells can be cancerous.

In some aspects, the plurality of endothelial cells can be obtained from the eye, kidney, brain, or another organ with endothelial tissues. In some aspects, the plurality of endothelial cells can be human.

In some aspects, the method can produce any of the disclosed biochips.

In another aspect, provided is a method of making a Schlemm's canal model, the method including: a) providing a substrate including a mechanical stressor; b) seeding a plurality of Schlemm's canal (SC) cells on top of the mechanical stressor; and c) inducing transcellular pore formation in the plurality of SC cells using the mechanical stressor. In some aspects, transcellular pore formation can be induced by local cellular stretch over the mechanical stressor.

In some aspects, the method can further include: d) exposing the plurality of SC cells to a tracer, wherein the tracer can bind to the substrate (e.g., the substrate or a coating on the substrate) at any regions of said substrate not covered by the plurality of SC cells; and e) detecting the tracer, thereby determining the presence of transcellular pores in the plurality of SC cells.

In some aspects, the tracer can include a fluorophore or fluorescent dye. In some such aspects, the tracer can include fluorescently labeled streptavidins (FL-SA).

In some aspects, the substrate can include glass. In other aspects, the substrate can include a hydrogel (e.g., a PEG hydrogel, Matrigel, etc.). In yet other aspects, the substrate can include glass at least partially covered by a hydrogel. In some aspects, the substrate can include a biocompatible material that can be mechanically deformed.

In some aspects, the substrate can further include a coating. In some such aspects, the coating can include biotinylated, crosslinked gelatin. Additionally or alternatively, in other such aspects, the coating can be configured to bind to a tracer at any regions of said coating not covered by the plurality of endothelial cells. Additionally or alternatively, in yet other such aspects, the coating can induce or enhance local stress over the mechanical stressor.

In some aspects, the mechanical stressor can include a plurality of ferromagnetic particles. In some such aspects, the plurality of ferromagnetic particles can include carboxyl-coated ferromagnetic particles.

In some aspects, each of the plurality of ferromagnetic particles can have a diameter of at least about 1 μm (e.g., at least about 1.5 μm, at least about 2 μm, at least about 2.5 μm, at least about 3 μm, at least about 3.5 μm, at least about 4 μm, at least about 4.5 μm, at least about 5 μm, at least about 5.5 μm, at least about 6 μm, at least about 6.5 μm, at least about 7 μm, at least about 7.5 μm, at least about 8 μm, at least about 8.5 μm, at least about 9 μm, at least about 9.5 μm, at least about 10 μm). In some aspects, each of the plurality of ferromagnetic particles can have a diameter of up to about 10 μm (e.g., up to about 9.5 μm, up to about 9 μm, up to about 8.5 μm, up to about 8 μm, up to about 7.5 μm, up to about 7 μm, up to about 6.5 μm, up to about 6 μm, up to about 5.5 μm, up to about 5 μm, up to about 4.5 μm, up to about 4 μm, up to about 3.5 μm, up to about 3 μm, up to about 2.5 μm, up to about 2 μm, up to about 1.5 μm, up to about 1 μm). In some aspects, each of the plurality of ferromagnetic particles can have a diameter of about 1 μm, about 1.5 μm, about 2 μm, about 2.5 μm, about 3 μm, about 3.5 μm, about 4 μm, about 4.5 μm, about 5 μm, about 5.5 μm, about 6 μm, about 6.5 μm, about 7 μm, about 7.5 μm, about 8 μm, about 8.5 μm, about 9 μm, about 9.5 μm, or about 10 μm.

It is considered that each of the plurality of ferromagnetic particles can have a diameter ranging from any of the minimum values described above to any of the maximum values described above. For example, in some aspects, each of the plurality of ferromagnetic particles can have a diameter of from about 1 μm to about 10 μm (e.g., from about 1.5 μm to about 9.5 μm, from about 2 μm to about 9 μm, from about 2.5 μm to about 8.5 μm, from about 3 μm to about 8 μm, from about 3.5 μm to about 7.5 μm, from about 4 μm to about 7 μm, from about 4.5 μm to about 6.5 μm, from about 5 μm to about 6 μm, from about 1 μm to about 5.5 μm, from about 1.5 μm to about 5 μm, from about 2 μm to about 4.5 μm, from about 2.5 μm to about 4 μm, from about 3 μm to about 3.5 μm, from about 5.5 μm to about 10 μm, from about 6 μm to about 9.5 μm, from about 6.5 μm to about 9 μm, from about 7 μm to about 8.5 μm, from about 7.5 μm to about 8 μm).

In some aspects, each of the plurality of ferromagnetic particles can have approximately the same diameter (e.g., from about 1 μm to about 10 μm as described above) to yield pores of approximately equal sizes. In other aspects, each of the plurality of ferromagnetic particles can have different diameters (e.g., from about 1 μm to about 10 μm as described above) to yield pores of varying sizes.

In some aspects, step c) can further include applying a magnetic force to the plurality of SC cells. In some aspects, the magnetic force can be at least about 400 N (e.g., at least about 450 N, at least about 500 N, at least about 550 N, at least about 600 N, at least about 650 N, at least about 700 N, at least about 750 N, at least about 800 N, at least about 850 N, at least about 900 N, at least about 950 N, at least about 1000 N, at least about 1050 N, at least about 1100 N, at least about 1150 N, at least about 1200 N, at least about 1250 N, at least about 1300 N, at least about 1350 N, at least about 1400 N, at least about 1450 N, at least about 1500 N). In some aspects, the magnetic force can be up to about 1500 N (e.g., up to about 1450 N, up to about 1400 N, up to about 1350 N, up to about 1300 N, up to about 1250 N, up to about 1200 N, up to about 1150 N, up to about 1100 N, up to about 1050 N, up to about 1000 N, up to about 950 N, up to about 900 N, up to about 850 N, up to about 800 N, up to about 750 N, up to about 700 N, up to about 650 N, up to about 600 N, up to about 550 N, up to about 500 N, up to about 450 N, up to about 400 N). In some aspects, the magnetic force can be about 400 N, about 450 N, about 500 N, about 550 N, about 600 N, about 650 N, about 700 N, about 750 N, about 800 N, about 850 N, about 900 N, about 950 N, about 1000 N, about 1050 N, about 1100 N, about 1150 N, about 1200 N, about 1250 N, about 1300 N, about 1350 N, about 1400 N, about 1450 N, or about 1500 N.

It is considered that the magnetic force can range from any of the minimum values described above to any of the maximum values described above. For example, in some aspects, the magnetic force can be from about 400 N to about 1500 N (e.g., from about 450 N to about 1450 N, from about 500 N to about 1400 N, from about 550 N to about 1350 N, from about 600 N to about 1300 N, from about 650 N to about 1250 N, from about 700 N to about 1200 N, from about 750 N to about 1150 N, from about 800 N to about 1100 N, from about 850 N to about 1050 N, from about 800 N to about 1000 N, from about 850 N to about 950 N, from about 400 N to about 900 N, from about 450 N to about 850 N, from about 500 N to about 800 N, from about 550 N to about 750 N, from about 600 N to about 700 N, form about 900 N to about 1500 N, from about 950 N to about 1450 N, from about 1000 N to about 1400 N, from about 1050 N to about 1350 N, from about 1100 N to about 1300 N, from about 1150 N to about 1250 N).

In some aspects, the magnetic force can be applied to the plurality of SC cells for at least about 1 minute (e.g., at least about 2 minutes, at least about 3 minutes, at least about 4 minutes, at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 60 minutes). In some aspects, the magnetic force can be applied to the plurality of SC cells for up to about 60 minutes (e.g., up to about 55 minutes, up to about 50 minutes, up to about 45 minutes, up to about 40 minutes, up to about 35 minutes, up to about 30 minutes, up to about 25 minutes, up to about 20 minutes, up to about 15 minutes, up to about 10 minutes, up to about 5 minutes, up to about 4 minutes, up to about 3 minutes, up to about 2 minutes, up to about 1 minute). In some aspects, the magnetic force can be applied to the plurality of SC cells for about 1 minute, about 2 minutes, about 3 minutes, about 4 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes.

It is considered that the magnetic force can be applied to the plurality of SC cells for a duration ranging from any of the minimum values described above to any of the maximum values described above. For example, in some aspects, the magnetic force can be applied to the plurality of SC cells for from about 1 minute to about 60 minutes (e.g., from about 2 minutes to about 55 minutes, from about 3 minutes to about 50 minutes, from about 4 minutes to about 45 minutes, from about 5 minutes to about 40 minutes, from about 10 minutes to about 35 minutes, from about 15 minutes to about 30 minutes, from about 20 minutes to about 25 minutes, from about 1 minute to about 25 minutes, from about 2 minutes to about 20 minutes, from about 3 minutes to about 15 minutes, from about 4 minutes to about 10 minutes, from about 20 minutes to about 60 minutes, from about 25 minutes to about 55 minutes, from about 30 minutes to about 50 minutes, from about 35 minutes to about 45 minutes).

In other aspects, the mechanical stressor can include a plurality of nonmagnetic nanoparticles and/or microparticles. In some such aspects, the mechanical stressor can include a plurality of ferromagnetic particles and a plurality of nonmagnetic nanoparticles and/or microparticles. In yet other aspects, the mechanical stressor can include a plurality of physical deformations (e.g., ridges, holes, bumps, etc.) in the substrate.

In some aspects, the plurality of SC cells can be healthy. In other aspects, the plurality of SC cells can be diseased or abnormal. For example, in some aspects, the plurality of SC cells can be glaucomatous. In other aspects, the plurality of SC cells can be consistent with another disease or disorder associated with abnormal (i.e., abnormally high or abnormally low) intraocular pressure (IOP).

In some aspects, the plurality of SC cells can be human. In some such aspects, the plurality of SC cells can be derived from a subject having glaucoma, cancer, or another disease or disorder associated with abnormal (i.e., abnormally high or abnormally low) intraocular pressure (IOP).

In some aspects, the method can produce any of the disclosed biochips.

In another aspect, provided is a method including: coating a glass substrate with biotinylated gelatin cross-linked with microbial transglutaminase; seeding the coated glass substrate with carboxyl ferromagnetic particles and, subsequently, primary human Schlemm's canal (SC) cells; adding a first tracer to the coated and seeded glass substrate; washing the first tracer from the coated and seeded glass substrate after a first time period; adding a second tracer to the coated and seeded glass substrate; applying a magnetic force to the coated and seeded glass substrate to create local cellular stretch in the primary human SC cells due to the carboxyl-coated ferromagnetic particles; and imaging the primary human SC cells to identify tracer signals.

In some aspects, the glass substrate can be a glass multiwell plate.

In some aspects, the first time period can be five minutes.

In some aspects, the method can further include incubating the coated and seeded glass substrate for a second time period prior to adding the first tracer.

In some aspects, the second time period can be between five and seven hours.

In some aspects, the primary human SC cells can be imaged at least twenty minutes after the second tracer was added.

In some aspects, the primary human SC cells can include two normal SC cell strains and one glaucomatous SCL call strain.

In some aspects, the first and second tracers can be streptavidin.

In some aspects, the local cellular stretch in the primary human SC cells due to the magnetic field can be in the basal-to-apical direction.

In yet another aspect, provided is a method of screening agents that modulate transcellular pore formation, the method including: i) providing an endothelial barrier model prepared by any of the disclosed methods; ii) exposing the endothelial barrier model to a therapeutic agent; and iii) determining the presence of transcellular pores in the plurality of endothelial cells as compared to an endothelial barrier model not exposed to the therapeutic agent.

In some aspects, the therapeutic agent can increase the formation of transcellular pores as compared to an endothelial barrier model not exposed to the therapeutic agent. In other aspects, the therapeutic agent can decrease the formation of transcellular pores as compared to an endothelial barrier model not exposed to the therapeutic agent.

In some aspects, step ii) can occur before inducing transcellular pore formation in the plurality of endothelial cells using the mechanical stressor. Additionally or alternatively, in other aspects, step ii) can occur during inducing transcellular pore formation in the plurality of endothelial cells using the mechanical stressor. Additionally or alternatively, in yet other aspects, step ii) can occur after inducing transcellular pore formation in the plurality of endothelial cells using the mechanical stressor.

In yet another aspect, provided is a method of screening agents that modulate transcellular pore formation, the method including: i) providing a Schlemm's canal model prepared by any of the disclosed methods; ii) exposing the Schlemm's canal model to a therapeutic agent; and iii) determining the presence of transcellular pores in the plurality of SC cells as compared to a Schlemm's canal model not exposed to the therapeutic agent.

In some aspects, the therapeutic agent can increase the formation of transcellular pores as compared to a Schlemm's canal model not exposed to the therapeutic agent. In other aspects, the therapeutic agent can decrease the formation of transcellular pores as compared to a Schlemm's canal model not exposed to the therapeutic agent.

In some aspects, step ii) can occur before inducing transcellular pore formation in the plurality of SC cells using the mechanical stressor. Additionally or alternatively, in other aspects, step ii) can occur during inducing transcellular pore formation in the plurality of SC cells using the mechanical stressor. Additionally or alternatively, in yet other aspects, step ii) can occur after inducing transcellular pore formation in the plurality of SC cells using the mechanical stressor.

### EXAMPLES

### Example 1: Biomechanically-Induced Transcellular Pores: A Screening Platform for Discovering Glaucoma Treatments

Transcellular pore formation regulates transport of cells and fluids across endothelial barriers. In Schlemm's canal (SC) endothelium, impaired pore formation is associated with glaucoma, a leading cause of blindness. However, the understanding of the cellular processes responsible for pore formation is limited by a lack of suitable in vitro assays. A study presented the first in vitro platform for transcellular pore formation in primary human SC cells. Pore formation was induced by seeding cells atop micron-sized magnetic beads placed within a magnetic field to apply an apical-directed forces to the basal cell surface so as to mimic the biomechanical loading experienced by SC cells in vivo. Glaucomatous cells exhibited impaired pore formation, associated with increased cell stiffness. Likewise, compounds that reduced cell stiffness promoted pore formation and compounds that increased cell stiffness reduced pore formation. Overall, the study provides a new approach to study and screen for transcellular pore formation in endothelial cells, revealing a key role of cellular biomechanical stiffness.

Transcellular transfer of materials (e.g., cells, proteins, or fluids) directly across cellular barriers occurs in situations such as leukocyte transmigration through microvascular endothelial cells,¹⁻⁷ glomerular filtration across endothelial cells,⁸ and drainage of aqueous humor (AH) from the eye via pores within Schlemm's canal (SC) inner wall endothelial cells.⁹⁻¹² In these cases, a pore (opening) must be created through an endothelial cell without damaging the cell. Understanding of the cellular processes responsible for such pore formation is limited, in part due to the small size of these pores and in part due to a lack of suitable in vitro assays replicating the pore formation process. Recent discoveries showing that SC pore formation is a mechanosensitive process and that pore formation can be facilitated by mechanical stretching¹³ and transendothelial perfusion¹⁴ motivate the development of discovery tools to study the mechanism(s) of mechanosensitive pore formation.

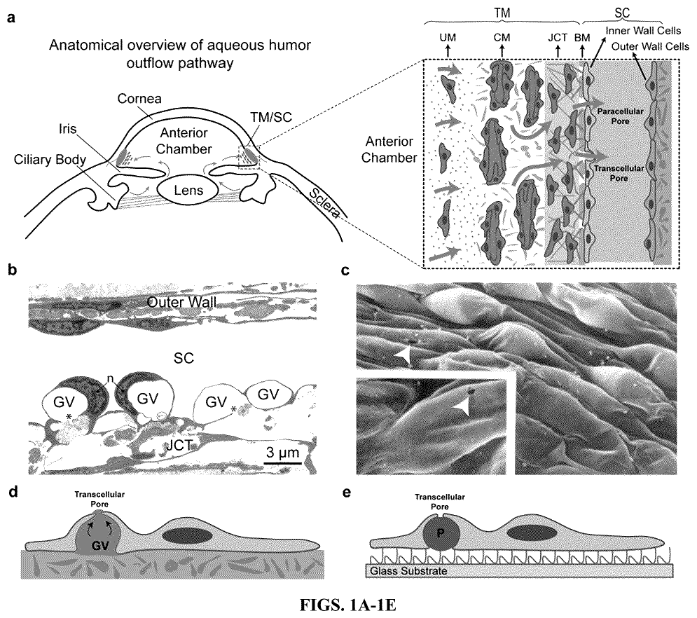

A key application for such tools is developing therapies to treat glaucoma, the leading cause of irreversible blindness¹⁵ affecting over 70 million individuals globally.^{16,17} All existing medical and surgical treatments for glaucoma aim to lower intraocular pressure (IOP) to decelerate disease progression,^{18,19} yet existing approaches are insufficient.²⁰ IOP is strongly influenced by AH transport through micron-sized pores that form in endothelial cells lining inner wall of SC, the only continuous cellular barrier within the major pathway for drainage of AH from the eye²¹ (FIG. 1A). Two types of pores form in SC cells and participate in fluid drainage from the eye: transcellular and paracellular pores¹² (FIG. 1A). Pores are triggered by cellular deformation due to the significant deformation experienced by SC endothelial cells in response to the normal transcellular basal-to-apical pressure drop²² in vivo (FIGS. 1B-1C).

SC pores are reduced in glaucoma,^{23,24} yet no current medications is shown to restore this compromised functionality,²⁵ motivating the discovery of “SC-active agents” for IOP control. Further, there is a lack of understanding of the molecular participants in the pore forming process. A study was conducted which developed an in vitro platform enabling generation and detection of transcellular pores in cultured primary human SC cells. This platform can be used for studies on the mechanobiology of pore formation and for screening of agents that promote pore formation, thereby lowering IOP.

### Methods

Isolation and characterization phSC cells: The protocols involving the use of human tissue adhered to the tenets of the Declaration of Helsinki. phSC cells were isolated using the cannulation method from human donor eyes, and were then cultured and characterized as previously described.^{26,27} Characterization was based on the cell's typical spindle-like elongated cell morphology, expression of vascular endothelial-cadherin and fibulin-2, a net transendothelial electrical resistance of 10 ohms·cm² or greater, and the absence of myocilin induction following exposure to dexamethasone. A total of five cell strains isolated from five healthy donor eyes and three cell strains isolated from three donors having a history of glaucoma were used in this study (TABLE 1). The SC cells were cultured in low glucose Dulbecco's Modified Eagle Medium (DMEM; Gibco 11885084) supplemented with 10% fetal bovine serum (FBS), and 1× penicillin streptomycin glutamine (PSG; Gibco 10378016) at 37° C. with 5% CO₂.

TABLE 1
Summary of cell strains. This table presents information on cell strain ID, donor
age, and sex. The initial letter in the cell strain ID indicates whether the donor
had a history of glaucoma (g) or if the cells were obtained from normal (n) eyes.

| | | | | Facility (μL/ | Glaucoma | |
|---|---|---|---|---|---|---|
| ID | Age | | Sex | min × mm Hg) | Type | Medications |
| nSC74 | 8 | months | Male | — | — | — |
| nSC75 | 10 | years | Male | — | — | — |
| nSC82 | 56 | years | Unknown | — | — | — |
| nSC87 | 62 | years | Male | — | — | — |
| nSC89 | 68 | years | Male | — | — | — |
| gSC57 | 78 | years | Male | — | — | — |
| gSC64 | 78 | years | Unknown | 0.07 | Normal Tension | — |
| gSC90 | 71 | years | Female | 0.25 | — | Cospot and |
| | | | | | | Latanoprost |

Alternatively, in some experiments HUVECs (ATCC, PCS-100-010) or HDMVECs (ATCC, PCS-110-010) were used instead of phSC cells.

Gelatin biotinylation: A gelatin solution (Type B, 1% in H₂O, Sigma-Aldrich G1393) was dialyzed against 0.1 M NaHCO₃ at pH 8.3. The biotinylation reagent (EZ-Link™ NHS-LC-LC-Biotin, Thermo Scientific 21343, in dimethyl sulfoxide) was then mixed in 12-fold excess molarity with the gelatin solution at room temperature for 1 hour. Subsequently, the biotinylated gelatin solution underwent dialysis against phosphate buffer saline (PBS) to remove excess biotin. The purified biotinylated gelatin was aliquoted and stored at 4° C. or preserved at −20° C. for long-term storage.

Substrate preparation and cell culture for transcellular pore formation using microparticles: Round cover glasses (12 mm in diameter) were employed for SEM imaging, while 16-well chambered cover glasses (16 wells per cover glass; Invitrogen C37000) were utilized for other experiments. Glass substrates underwent plasma treatment (Harrick Plasma, PDC-32G) for 2 minutes at high RF power and were immediately incubated at 37° C. with 0.5 mg/mL biotinylated gelatin for 24 hours. After three washes with PBS, the cover glasses were incubated with microbial transglutaminase (0.001 unit/mL in 20 mM Tris-HCl and 150 mM NaCl, pH 8; Sigma-Aldrich SAE0159) at 37° C. for 24 hours to crosslink the gelatin coating and enhance phSC cell attachment on the substrate. Following three additional washes with PBS, the cover glasses were UV sterilized in a biosafety cabinet for 1 hour. Carboxyl ferromagnetic particles (4.0-4.9 μm, Spherotech, Inc. CFM-40-10) were vortexed and added to the substrate. The particles were allowed to randomly sink onto the substrate to achieve an approximate density of 1 particle per 1000 μm². Non-adherent particles were removed by washing three times with PBS, followed by seeding phSC cells (passage 4-6; 7.5 k cells/cm²). The cells were then cultured for 2 to 7 hours in DMEM supplemented with 1% FBS and 1×PSG at 37° C. with 5% CO₂. The culture time was chosen as the minimum duration necessary for the cells to adopt a spread configuration similar to that observed in situ. Note that this culture time (2 to 7 hours) varied for the tested cell strains. Alternatively, in some experiments, apical-to-basal cell stretching was achieved by adding particles above fully spread cells.

SEM sample preparation and imaging: Cells were fixed in universal fixative (2.5% glutaraldehyde, 2.4% paraformaldehyde, and 0.08 M Sorensen's phosphate buffer in PBS) for 30 minutes and washed three times with PBS. Following fixation, the cells were incubated in a 2% tannic acid and guanidine hydrochloride solution in the dark for 2 hours, and then washed three times in PBS over a 60-minute period. Subsequently, the cells were post-fixed in 1% osmium tetroxide in PBS for 60 minutes, washed three times with PBS, and then rinsed three times in ultra-pure water. The cells were then dehydrated in an ethanol series (25, 50, 75, 90, 100, 100, and 100%; 10-minute changes), transferred to a 1:1 mix of 100% ethanol: hexamethyldisilazane (HMDS) for 10 minutes, followed by two 10-minute changes in HMDS, and air-dried in a fume hood by vigorous shaking. Further drying of the cells was allowed overnight in a chemical hood. Subsequently, the cells were mounted on SEM stubs with conductive carbon adhesive tapes. Samples were coated with approximately 10 nm of gold/platinum using a spotter coater and examined with a Hitachi SU8010 Cold Field Emission SEM.

Pore detection using fluorescent assay and confocal microscopy: Once cells had fully spread over particles, they were incubated with the first pore label (5 μg/mL; Streptavidin, Alexa Fluor 488 Conjugate, Invitrogen S32354) for 5 minutes at 37° C. Subsequently, the cells were immediately washed three times with PBS, fixed with 4% formaldehyde for 15 minutes at room temperature, washed three times with PBS, permeabilized with 0.1% Triton X for 15 minutes, and stained with HCS CellMask Stains (10 μg/mL; Invitrogen H32712) for 1 hour. Following these steps, cells were washed three times with PBS and stored in PBS at 4° C. until examination with a Zeiss LSM 900 confocal microscope system.

High-content screening of transcellular pores: Upon achieving full cell spreading over particles, cells were incubated with the first pore label for 5 minutes, followed by three washes with cell media. Subsequently, cells were either exposed to a magnetic field (as detailed below) or incubated solely with cell media for 10 minutes. Afterward, the cells were incubated with the second pore label (5 μg/mL; Streptavidin, Alexa Fluor 647 Conjugate, Invitrogen S32357) for 5 minutes, all at 37° C. The cells were then immediately washed three times with PBS, fixed with 4% formaldehyde for 15 minutes at room temperature, washed three times with PBS, permeabilized with 0.1% Triton X for 15 minutes, and stained with NucBlue Fixed Cell Ready Probes Reagent (6 drops/mL; Invitrogen R37606) and Alexa Fluor 555 Phalloidin (0.165 μM; Invitrogen R34055) for 1 hour at room temperature. Following these steps, cells were washed three times with PBS, the cover glass was detached from the rest of the 16-well chambered system, mounted on another cover glass using Prolong Gold Antifade Mountant (Invitrogen P10144), and stored in the dark until examination with a Leica DM6 B upright microscope system.

Fluorescent and brightfield images were acquired using a 10× objective to generate tile scans for each well. To exclude cells near the well edges, a 4 mm diameter circle was delineated at the center of each image, and the analysis was restricted to the cells within this 4 mm circle. The number of cells, particles beneath cells, and pores were manually counted. Pores incidence rate was quantified as the number of pores associated with particles divided by the number of particles, multiplied by 100.

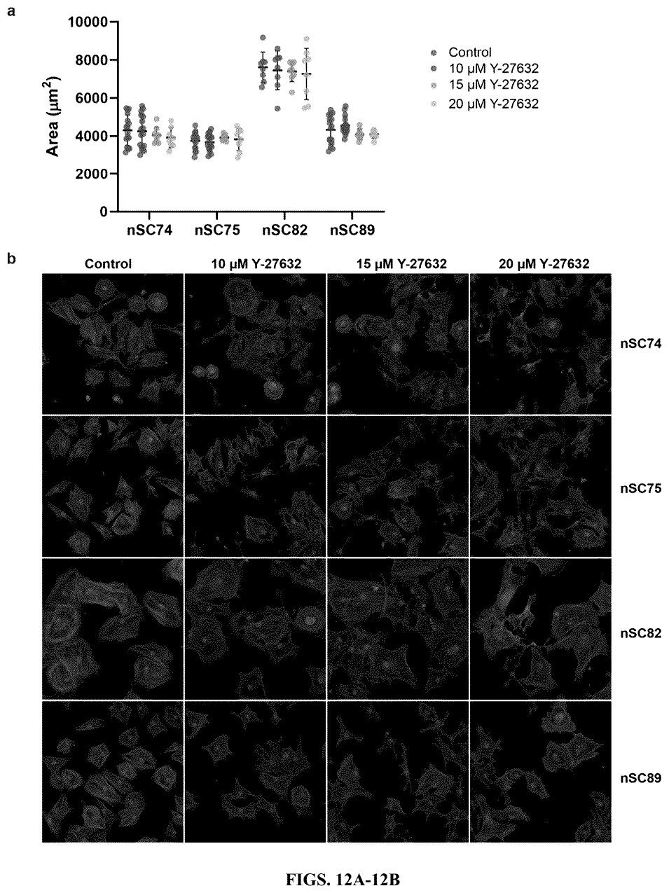

Magnetic field application and measurement: Magnetic microparticles were exposed to a magnetic field for 10 minutes between the first and second pore label. Utilizing a custom-designed magnetic actuator, neodymium N52 cylindrical magnets ( 3/16″ diameter×½″ thick, D38-N52, K&J Magnetics) were precisely positioned in each well of the 16-well chambered slides at approximately 0.20 mm (high force; estimated 461±10 pN on each particle), 0.55 mm (medium force; 337±11 pN), or 0.91 mm (low force; 235±9 pN) above the cells (FIGS. 11A-11G). Alternatively, in experiments where magnetic particles were situated atop cells to induce local apical-to-basal cell deformation, D38-N52 magnets were arranged within a well-plate, with the 16-well chambered slides directly positioned atop the magnets. In this setup, magnets were separated from the particles by a distance of 0.17 mm (equivalent to the thickness of the #1.5 coverslip), generating a magnetic force of 473±11 pN per particle.

As shown in FIGS. 11A-11G, the magnetic force exerted on individual particles was assessed by positioning magnets within a well-plate atop a calibrated scale, with the scale zeroed. Employing a high-precision Z-axis stage (Motorized Vertical Translation Stage 8MVT188-20), a coverslip was delicately situated slightly above the magnets, ensuring no direct contact. A known quantity of particles was then pipetted above the magnets, and the scale was read to calculate the axial force applied on the particles. Through incremental adjustments to the distance between the magnets and particles utilizing the Z-axis stage, the magnetic force acting on particles at various distances from the magnet surface was measured (n=3 repeats). Given that the magnetic force opposed the gravitational force on the magnets registered by the scale, alterations in the scale reading were utilized to determine the total magnetic force applied to the particles/magnets at specified distances. The magnetic force exerted on each particle was subsequently calculated by dividing the total force by the number of particles.

Treatment with Y-27632 ROCK inhibitor: Cells were seeded above microparticles in 16-well chambered slides in DMEM supplemented with 10% FBS, 1×PSG and allowed to spread for 3.5 hours at 37° C. with 5% CO₂. Subsequently, the cells were incubated with 10 μM Y-27632 (Sigma, Y0503) in DMEM supplemented with 1% FBS, 1×PSG for 30 minutes, followed by the first pore label for 5 minutes. Following incubation, the cells were washed, fixed, stained for nuclei and actin filaments, and imaged as described in the section titled “High-content screening of transcellular pores.”

Cell stiffness measurement: SC cells were seeded on coverslips and grown to confluence. An MFD-3D AFM (Asylum Research, Santa Barbara, CA, USA) was used to make stiffness measurements using silicon nitride cantilevers with an attached borosilicate sphere (diameter=10 μm; nominal spring constant=0.1 N/m; Novascan Technologies, Inc., Ames, IA, USA). Cantilevers were calibrated by measuring the thermally induced motion of the unloaded cantilever before measurements. The indentation depth was limited to 400 nm to avoid substrate effects and the tip velocity was adjusted to 800 nm/s to avoid viscous effects³⁷. 5 measurements/cell were conducted, and at least 5 cells were measured/cell strain. Data from AFM measurements were fitted to the Hertz model to calculate the Young's Modulus of the cells.

Statistical analysis: Statistical analysis was conducted using GraphPad Prism, with significance set at p<0.05. The mean and standard deviation were calculated, and the Shapiro-Wilk normality test was applied. For data passing the normality test, t-tests or ANOVA tests followed by Tukey's multiple comparison tests were employed. In cases where normality was not met, non-parametric Mann-Whitney tests or Kruskal-Wallis tests followed by Dunn's multiple comparisons tests were performed. Significance levels in figures are denoted by (ns), (*), (**), (***), and (****) for not significant, p<0.05, p<0.01, p<0.001, and p<0.0001, respectively.

### Results

Local basal-to-apical cellular stretch induces transcellular pore formation in SC cells: Primary human SC (phSC) cells were isolated, cultured and characterized from post mortem donor eyes as previously described.^{26,27} Previous research indicates that seeding phSC cells on top of micron-sized particles induces localized cellular deformation, mimicking the in vivo basal-to-apical biomechanical loading (FIGS. 1D-1E). This, in turn, initiates the formation of transcellular pores. ˜5 μm, carboxyl-coated, ferromagnetic microspheres were randomly seeded on gelatin-coated glass substrates, followed by seeding phSC cells on top of these particles (see Methods). Cells were then cultured for 2 to 7 hours so that they adopted a spread configuration similar to that seen in situ.

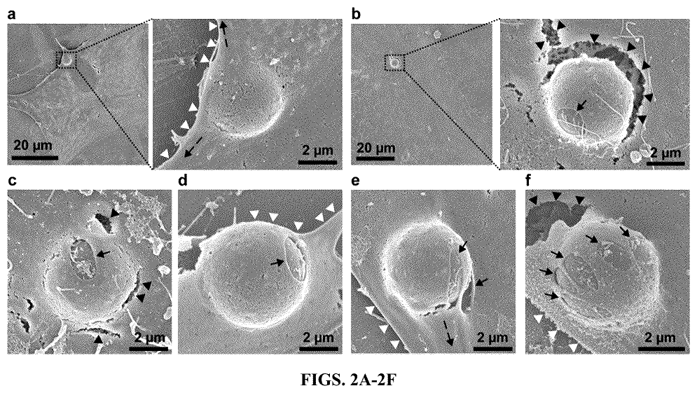

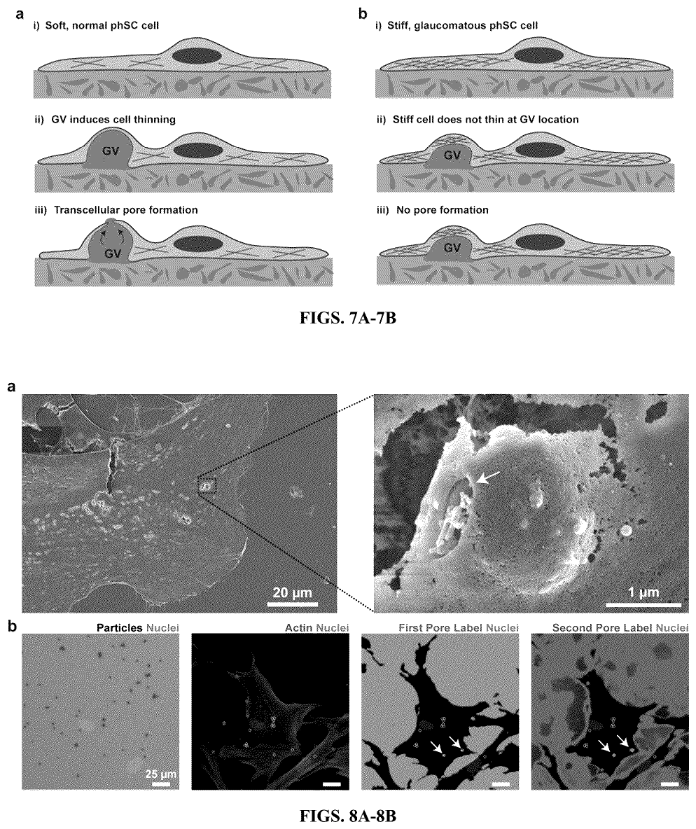

Scanning electron micrographs of fixed cells confirmed that phSC cells stretched over particles (FIG. 2A), with transcellular pores appearing above some particles (FIG. 2B). Notably, these transcellular pores exhibited smooth edges, allowing for clear differentiation from artifacts characterized by irregular, jagged edges that were likely byproducts of the scanning electron microscopy (SEM) sample preparation process.³⁰ Using high resolution SEM images, cell membrane thickness at the smooth edge of pores were measured as 56±11 nm (N=15 cells). Note that transcellular pores were also formed at the location of smaller (˜2 μm) particles (FIGS. 8A-8B), however only 5 μm particles were used in the rest of the study to facilitate detection of particles.

A significant diversity of transcellular pore locations and morphologies was observed. For example, some pores formed directly above (FIG. 2C) or adjacent to (FIG. 2D) particles. Further, it was observed that particles could induce pore formation irrespective of their specific location beneath cells; for example, transcellular pores formed in cells both with particles situated away from (FIG. 2B and FIG. 2C) and near (FIG. 2D and FIG. 2E) the cell border. In many cases, distinct artifactual cracks were observed, particularly adjacent to the particles (FIG. 2B and FIG. 2C), which is due to the cells stretching over the particles, rendering them more vulnerable to artifacts during the SEM sample preparation process.³⁰ However, there were instances (FIG. 2E) where the cell did not exhibit major artifacts adjacent to a particle despite noticeable cellular deformation. In the case shown in FIG. 2E, transcellular pores were formed to the side of the particle and extended along the direction of membrane stretching. Further, some particles induced the formation of only one transcellular pore (FIGS. 2B-2D), while others induced multiple transcellular pores (FIGS. 2E-2F). Of note, the transcellular pores shown in FIG. 2F exhibit a structure reminiscent of “toffee-like” pores previously reported by Braakman et al.¹³.

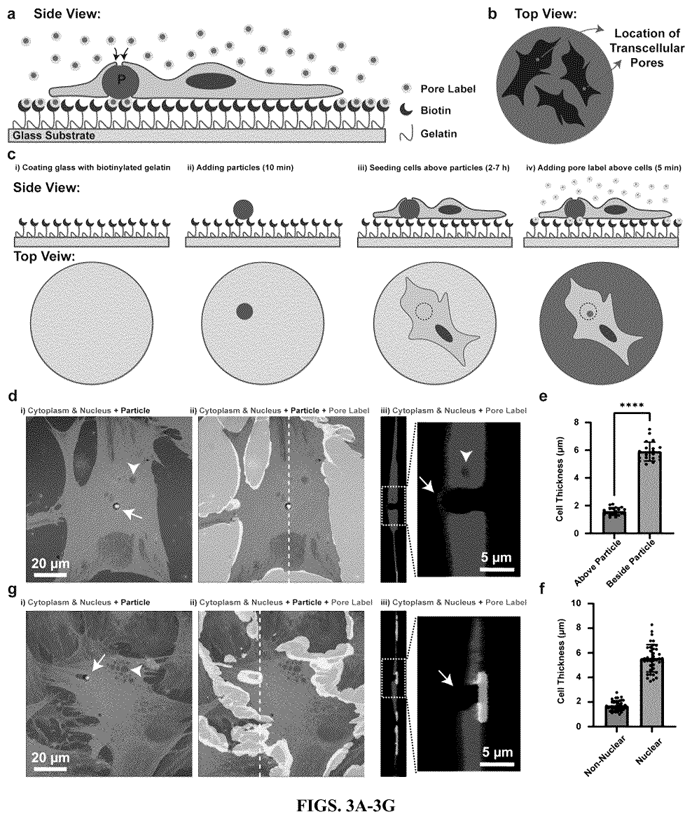

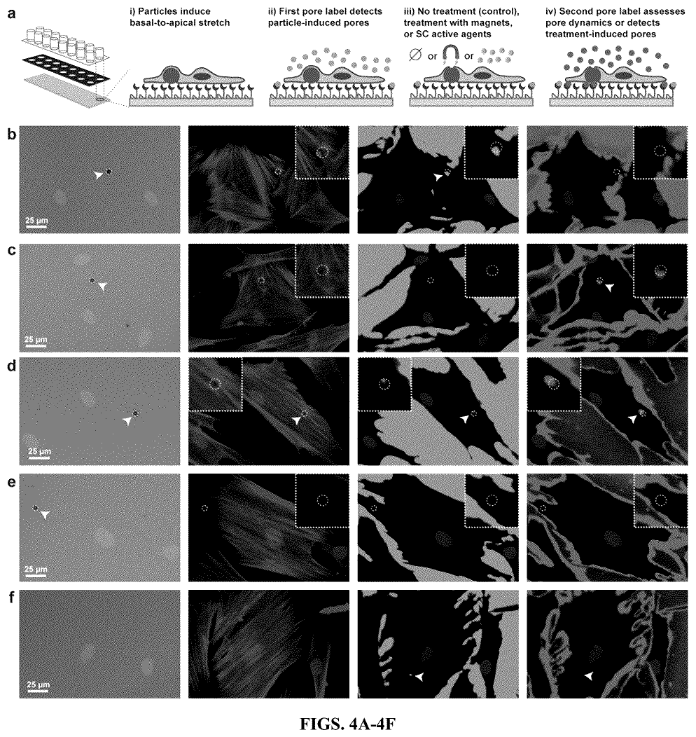

Transcellular pores are rapidly detectable using an in vitro fluorescent assay: Confocal microscopy and an established fluorescent assay^{31,32} (FIGS. 3A-3G) were used to further investigate cell deformation at the location of particles and to also rapidly identify transcellular pores without the need for time-consuming SEM imaging. In this assay, a pore label (fluorescently labeled streptavidin) adheres to a biotinylated gelatin substrate at sites not obscured by cells, marking areas surrounding subconfluent cells and, notably, at transcellular pore locations (FIGS. 3A-3B).

FIG. 3C illustrates the principle of the fluorescent assay to detect transcellular pores. High-definition confocal images of a representative cell overlying a ˜5 μm particle are shown in FIG. 3D. After seeding over particles, cells naturally deformed, thinning directly above the particle (white arrow in panel iii inset). Cell thickness was quantified using cytoplasmic and nuclear staining in cross-sectional views, revealing that cell thickness above particles (1.6±0.3 μm) was significantly less than beside the particles (5.9±0.7 μm; two-tailed, paired t test: p-value <0.0001; FIG. 3E). Cell thickness at nuclear (5.6±1.1 μm) and non-nuclear (1.7±0.4 μm) regions (FIG. 3F) was also measured which were on par with cell thickness beside and above particles, respectively.

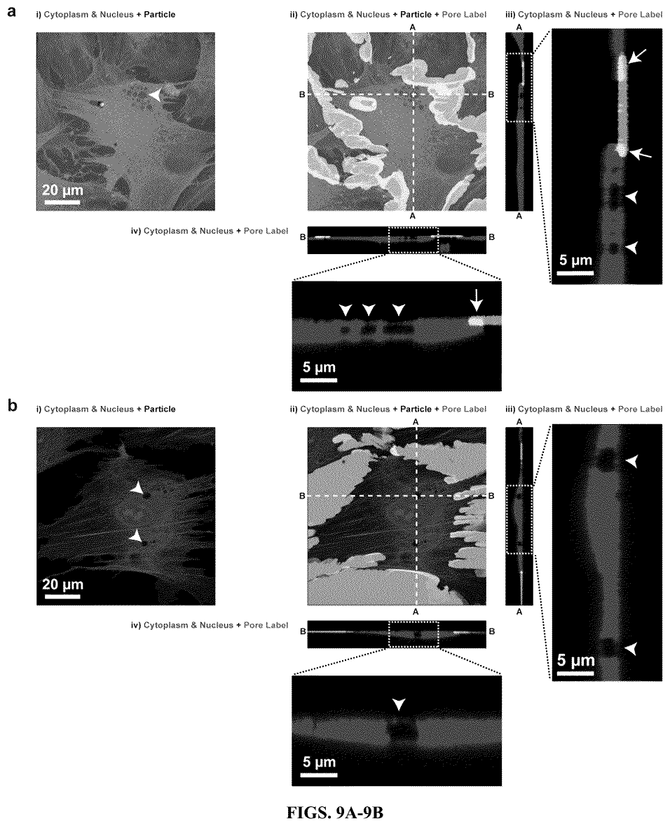

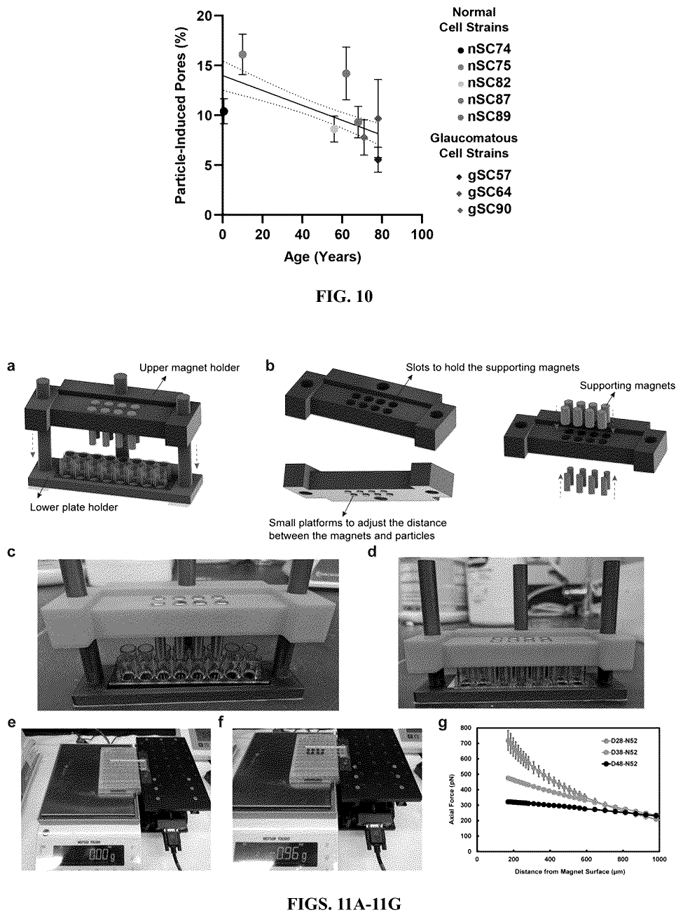

FIG. 3D displays a thinned region of a phSC cell stretched over a microparticle. Notably, the pore label did not adhere to the substrate near this microparticle (FIG. 3D, middle two panels), indicating the absence of pore formation. In contrast, in other cells (FIG. 3G), a cellular opening (transcellular pore) overlying the particle was observed, with the pore label also adhering to the substrate, indicating transcellular pore formation. Please note that in these confocal images, intracellular vesicles appear as empty spaces after cytoplasmic staining (white arrowheads in FIG. 3D and FIG. 3G). These vesicles are not openings extending through the cell, unlike transcellular pores, and were also observed in cells not positioned over particles (FIG. 9A-9B).

The SC “inner wall on a chip” allows high-content fluorescence detection of transcellular pores: The originally developed fluorescent assay coupled with confocal microscopy only allowed for the detection of particle-induced transcellular pores in a small number of cells. This limitation prevented high-throughput screening of compounds that facilitate pore formation in cells. Thus, the assay was expanded to analyze thousands of cells within hours, thereby enabling high-content fluorescence detection of transcellular pores.

The high-throughput assay allows for the detection of different types of transcellular pores. The assay effectively quantifies two major categories of transcellular pores: those induced by particles alone and those induced by subsequent treatments at the location of particles, such as exposure to an external magnetic field or active SC agents. For visualization, cells were seeded over particles in 16-well chambered slides and first exposed to a green pore label to detect particle-induced pores (FIG. 4A). These pores, observed after cells were over particles for 2-7 hours, represent the sustained impact of particles on pore formation. Following the initial labeling, cells underwent treatment and were then exposed to a second pore label, enabling the detection of treatment-induced pores. Images were captured using widefield microscopy (brightfield and fluorescence), which facilitated the examination of hundreds of cells per well (581±189 cells/well).

Pores were classified based on their response to the two fluorescent labels used in the assay. Transient pores were only detected by the first pore label and had closed at the time of the second labeling (FIG. 4B). Conversely, emergent pores appeared solely in response to the second pore label, indicating that they had formed after the initial labeling period (FIG. 4C). Persistent pores, observed with both pore labels, remained open throughout the labeling process, evidence of ongoing pore activity (FIG. 4D). Additionally, some particles did not induce any detectable pores with either label (FIG. 4E). Lastly, pores that were detected by the fluorescent assay but were not associated with any particle were occasionally observed (FIG. 4F). These results underscore the assay's capability to distinguish between various types of pore formation dynamics and their responses to different treatments, which is critical for advancing therapeutic strategies for glaucoma.

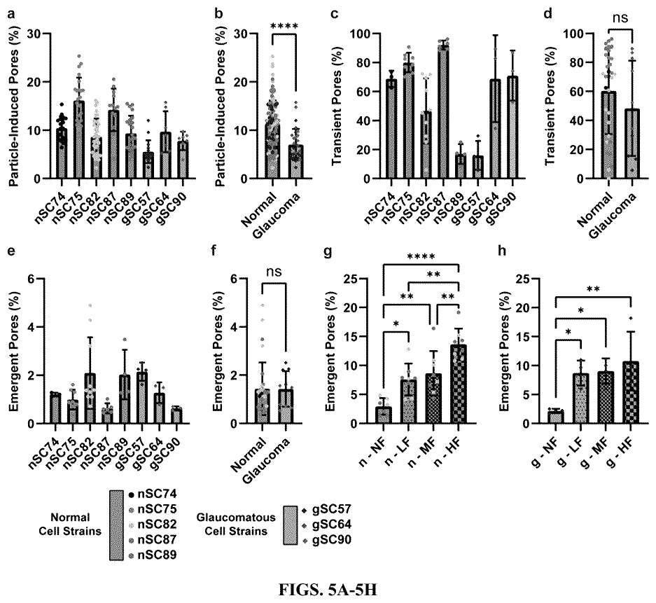

Particles induce fewer transcellular pores in glaucomatous cells vs. normal phSC cells: To determine if pores in the high-throughput assay were reduced as SC pores are in glaucoma^{23,24}, particle-induced pore counts in in normal and glaucomatous phSC cell strains were compared (FIG. 5A). Significant variability in particle-induced pore formation rate between the tested cell strains was observed, but pooling the data for normal and glaucomatous cell strains (FIG. 5B) indicated that the glaucomatous cell strains formed significantly less transcellular pores at the particle location relative to normal cells (7.0±3.2 vs. 11.0±5.0% pore/particle; two-tailed Mann-Whitney test: p-value <0.0001). Note that the tested glaucomatous cells were obtained from older donors compared to the normal cells and there was a weak correlation between particle-induced pore incidence and age of donors (R²=0.19, p<0.0001; FIG. 10)

The pore dynamics in normal and glaucomatous cells were further investigated by counting the percentage of pores detected by the first pore label but closed 10 minutes later when the cells were exposed to the second pore label without any treatments (transient pores; FIG. 5C). Pooling the data for normal and glaucomatous cells (FIG. 5D) demonstrated that the percentage of transient pores was not significantly different between normal and glaucomatous cells (60.4±29.8 vs. 48.3±32.8% pore; two-tailed Mann-Whitney test: p-value=0.3706).

Further, FIG. 5E shows the incidence of emergent pores, i.e. pores detected by the second pore label but not by the first pore label. Pooling the data (FIG. 5F) showed that the percentage of emergent pores was also not significantly different between normal and glaucomatous cells (1.4±1.1 vs. 1.4±0.7% pore/particle; two-tailed Mann-Whitney test: p-value=0.5053).

Magnetic force enhances transcellular pore formation in normal and glaucomatous phSC cells: An external magnetic field was applied to the magnetic microparticles in the dish, with the intention of promoting cell stretching, thus reducing the thickness of overlying cells and enhancing pore formation. Following the application of the magnetic field (see FIGS. 11A-11G), an increase in pore formation rates in both normal and glaucomatous cells was observed (FIGS. 5G-5H), even at the weakest magnetic field strength tested (leading to a force of 235±9 pN per particle). Interestingly, these pores increased 2.6-fold for normal cells (from 2.9±1.4 to 7.6±2.8; ANOVA p=0.0200) and 4.1-fold for glaucomatous cells (from 2.1±0.4 to 8.7±2.2; ANOVA p=0.0392) compared to the control group with no external force. Furthermore, at the highest magnetic field strength tested (461±10 pN per particle), magnet-induced emergent pores significantly increased, rising 4.7-fold for normal cells (from 2.9±1.4 to 13.6±2.7; ANOVA p<0.0001) and 5.0-fold for glaucomatous cells (from 2.1±0.4 to 10.8±5.1; ANOVA p=0.0070) compared to the control group. Therefore, magnetic force delivery enhances pore formation in the assay.

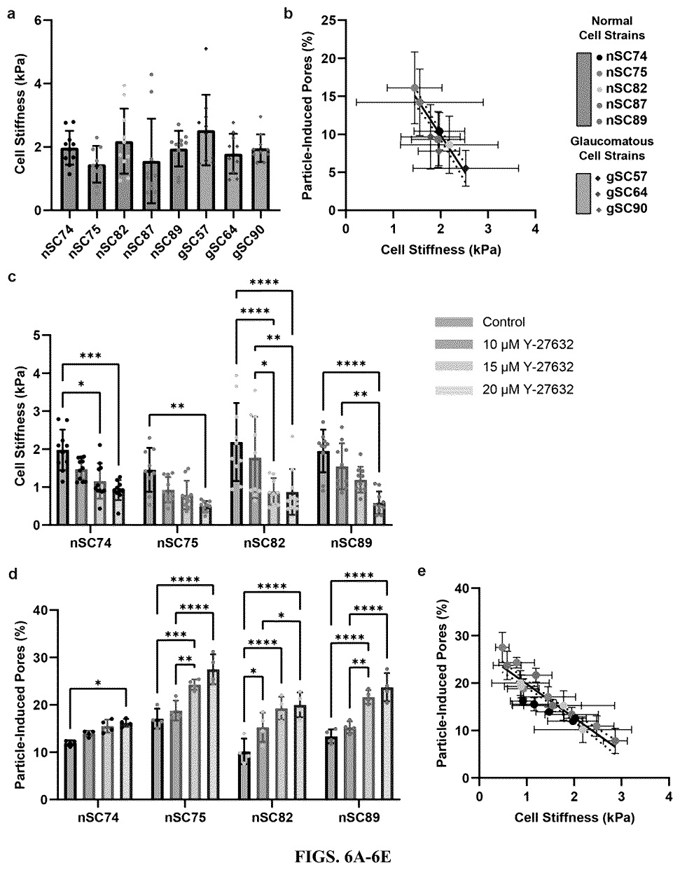

Pore formation is associated with phSC cell stiffness: Magnetic force enhances pore formation by reducing cell thickness at the particle location. Moreover, glaucomatous cells exhibited a greater response to the magnetic force compared to normal cells. Further, the inner wall of SC in glaucomatous eyes with elevated flow resistance is stiffer than that in normal eyes. 3.3 Based on these observations, pore formation is directly correlated with cell stiffness. Cell stiffness was measured using atomic force microscopy (AFM) on five normal and 3 glaucomatous cells strains (FIG. 6A). A regression analysis revealed an association between particle-induced pore incidence and cell stiffness (R²=0.41, p<0.0001). This indicates that stiffer cells have fewer particle-induced pores (FIG. 6B).

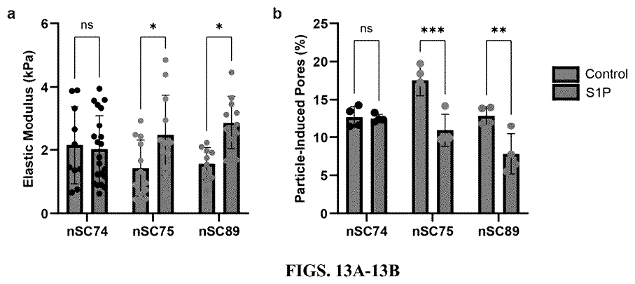

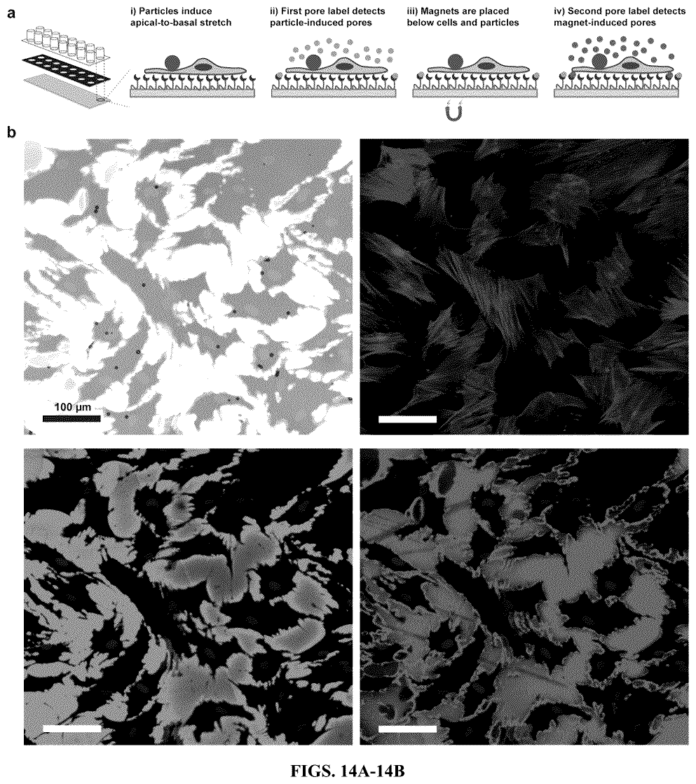

The ROCK inhibitor Y-27632 enhances transcellular pore formation in phSC cells: Pore formation is enhanced by lowering cell stiffness. Rho-associated kinase (ROCK) affects acto-myosin tone and cellular biomechanical properties, and ROCK inhibitors have emerged as a treatment for reducing IOP, primarily by reducing outflow resistance of AH via the relaxation of TM cells.³⁴ Thus, to the study investigated the impact of the effects of the ROCK inhibitor Y-27632 on pore formation. Y-27632 decreased cell stiffness (FIG. 6C) and increased pore formation in a dose-dependent manner in nSC74, nSC75, nSC82, and nSC89 (FIG. 6D). Note that the Y-27632 treatments did not significantly affect the cell area (FIGS. 12A-12B). Conversely, sphingosine-1-phosphate (SIP) increased cell stiffness and decreased pore formation in nSC75 and nSC89, however it did not have a significant effect on nSC74 (FIGS. 13A-13B). Of utmost significance, regression analysis unveiled a robust correlation between particle-induced pore incidence and cell stiffness (R²=0.74, p<0.0001) across four cell strains subsequent to altering cell stiffness through Y-27632 and SIP treatments.

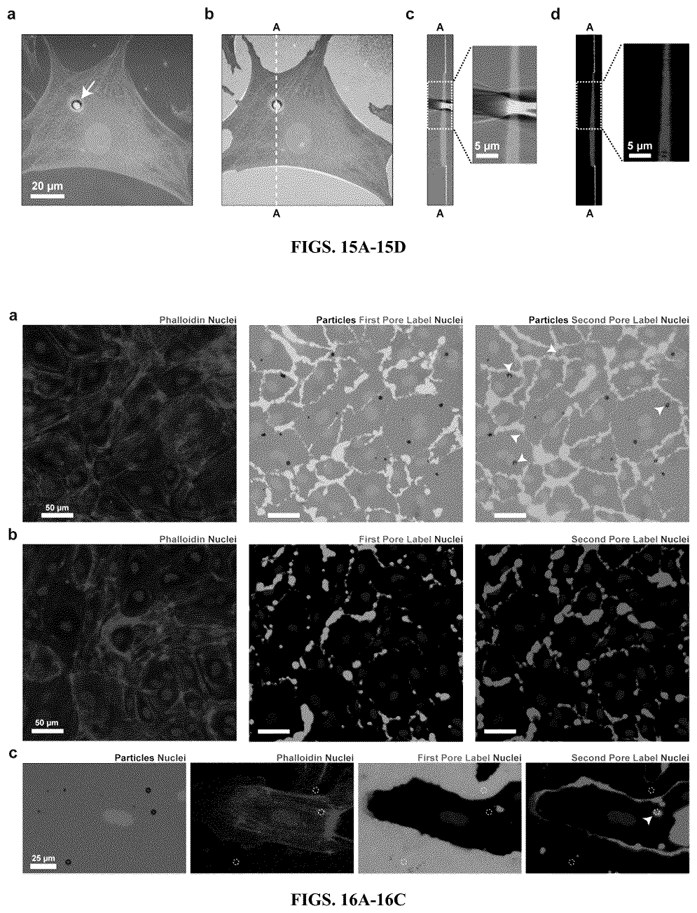

Particle-induced transcellular pores are directional: phSC cells experience basal-to-apical deformation in vivo and the study showed that seeding cells above particles induces transcellular pores in vitro (FIGS. 2A-2F, FIGS. 3A-3G, and FIGS. 4A-4F). When placing particles above phSC cells to apply an apical-to-basal local cellular deformation, did not result in any detectable transcellular pores using the fluorescent assay at the location of particles (FIGS. 14A-14B and FIGS. 15A-15D). Even applying a magnetic field (473±11 pN per particle; see Methods and FIGS. 11A-11G for magnetic force measurement) or treatment with 10 μM Y-27632 did not induce transcellular pores.

In diapedesis, leukocytes move directly through individual endothelial cells by forming transcellular routes from apical to basal direction,^{5,35} which is the opposite direction of AH transport through pores formed in endothelial cells lining the inner wall of SC. Thus, positioning particles atop cells capable of facilitating leukocyte transfer from the apical to basal direction in vivo prompted the formation of transcellular pores in the in vitro assay. Particles were positioned atop human umbilical vein endothelial cells (HUVECs) and human dermal microvascular endothelial cells (HDMVECs). And the formation of transcellular pores at the sites where particles were seeded on top of both HUVECs and HDMVECs (see FIGS. 16A-16C).

### DISCUSSION

The study describes the design, implementation, and validation of an in vitro platform for creating and detecting transcellular pores in adherent cells, with particular focus on transcellular pores in phSC cells. The platform is able to be used for high content screening of compounds that can modulate pore formation and ultimately improve the AH outflow facility for glaucoma management and treatment.

The in vitro assay successfully replicates important mechanobiological observations from whole tissue. By applying a locally relevant basal-to-apical cell stretching, the assay enables the creation and quantification of transcellular pores in both normal and glaucomatous phSC cells. Glaucomatous cells exhibited a generally impaired particle-induced pore formation, consistent with previously observed differences in pore formation between normal vs. glaucomatous SC cells, both in native tissues and in an in vitro assay inducing pore formation by a membrane stretching and perfusion device.^{13,14} The assay indicates that pore formation is triggered by mechanical stimulation (stretch). SEM micrographs show that pores form at the location of particles where cell membrane is stretched and thinned. Furthermore, a correlation between the focal force delivered magnetically and the formation of pores was established. Interestingly, the response to magnetic force was higher in glaucomatous cells compared to normal cells. When a minimal force of ˜250 pN per particle was applied, corresponding approximately to a local pressure by ˜0.1 mmHg over the surface of the particle, there was a significant 4.1-fold increase in pore formation for glaucomatous cells compared to a 2.6-fold increase for normal cells. The results suggest that impaired pore formation in glaucomatous cells can be rescued by further stretching and thinning these cells.

The study shows, for the first time, that the diminished pore formation in glaucomatous cells is attributed to increased cell stiffness, as evidenced by the close association observed between cell stiffness and pore formation rate. Additionally, the ROCK inhibitor Y-27632, a cell-softening agent, increased pore formation, while SIP, a cell-stiffening agent, decreased pore formation. These findings show that softer cells are more readily modifiable by particles, can be stretched and tinned above particles, and form transcellular pores. Currently no medication has been shown to directly target SC pores, but some, such as Netarsudil,³⁶ may affect SC pores since they improve fluid outflow through the trabecular meshwork by inhibition of Rho kinase.

This study creates a new model of transcellular pore formation in phSC cells (FIGS. 7A-7B). In this model, a relatively soft normal phSC cell experiences significant stretching at the location of GVs. Consequently, a transcellular pore forms at the thinnest location of the cell associated with GV (FIG. 7A). In contrast, the stiffer glaucomatous cells do not stretch and thin enough to initiate pore formation (FIG. 7B). The model indicates that to initiate pore formation, there is a minimum cell thickness that needs to be achieved.

The quantification of pores in the high-content studies was constrained by manual counting (i.e., identifying fluorescent signals at the particle locations). Pore identification becomes notably challenging when cells grow in close proximity. Given that phSC cells do not form tight junctions in vitro, the fluorescent tracer may leak between cells, reaching the substrate and generating false signals. Additionally, the lateral diffusion of the fluorescent tracer under cells (at pore locations) and/or cell movement may have caused the pores to appear larger than their actual size. The accuracy of the fluorescent assay could be enhanced by developing methods that enable the formation of tight junctions by phSC cells. Additionally, implementing computer-aided algorithms for pore identification could streamline and improve the overall process.

Overall, this study represents a significant advancement in the field of glaucoma research by providing a tool for studying the biophysical properties of SC cells and identifying therapeutic agents that can restore the compromised outflow facility. Elucidating the mechanisms underlying pore formation in SC allows for the development of innovative treatments targeting glaucoma and results in improving the quality of life for affected individuals.

### Example 2: Development of a Schlemm's Canal “Inner Wall on a Chip” for High Content Biomechanical Screening

A study developed a high content in vitro mechanobiological assay to assess I-pore formation in subconfluent SC cells exposed to focal stretch delivered by ferromagnetic microspheres. The study focuses on I-pores since SC cells do not form tight intercellular junctions in vitro; thus, methods for high-content screening of B-pores are more challenging.

### Methods

Glass substrates (Nunc™ Lab-Tek™ chamber slide system) were coated with 0.5 mg/ml biotinylated gelatin cross-linked with 1 unit/mL microbial transglutaminase. Carboxyl ferromagnetic particles (dia. ˜5 μm, Spherotech CFM-40-10) were seeded onto the substrate, followed by seeding with primary human SC cells (two normal SC cell strains: nSC89 and nSC82 and one glaucomatous cell strain: gSC57; all passage 5 or 6). Pores were detected using fluorescently labeled tracers [9]. Specifically, after 5-7 h of incubation, a first tracer (streptavidin, Alexa Fluor™ 488 conjugate) was added to the media for 5 min. The first tracer was then washed away, and a second tracer (streptavidin, Alexa Fluor™ 647 conjugate) was added for 20 min, during which time particles were exposed to a magnetic force created by neodymium N52 magnets (D38-N52, K&J Magnetics, Inc.) placed ˜0.20 mm (high force; estimated ˜1138 pN on each particle), ˜0.55 mm (medium force; ˜785 pN), or ˜0.91 mm (low force; ˜435 pN) above cells. The magnets attracted particles to create a local cellular stretch in the basal-to-apical direction in seeded SC cells. In some wells, magnets were not placed above cells as a negative (no force) control.

Cells were fixed, labeled with DAPI and Alexa Fluor™ 555 phalloidin, imaged, and analyzed by counting punctate tracer signals that colocalized with particles under cells. The number and area of labelled substrate under I-pores were quantified using custom MATLAB code. I-pores were categorized as Type I (first tracer, i.e., present without magnetic force/stretching) or Type II (second tracer, i.e., present with magnetic force/stretching). The number and area of Type II pores minus Type I pores (normalized by the number of particles under cells) were then computed as an indirect measure of porosity. The number of cells and particles analyzed for each cell strain were: nSC89: 4 wells per force condition; 404±78 cells/well, 106±51 particle/well; nSC82: 4 wells; 71±19 cells/well, 115±39 particle/well; and gSC57: 8 wells; 203±51 cells/well, 130±39 particle/well. Two-way ANOVAs were performed with factors: (i) force level (control, low, medium, high); and (ii) disease state of the cell strains (normal, glaucomatous). Outcome measures were the number and the area of detected pores. When ANOVA indicated a significant factor effect, outcomes were compared pairwise using Tukey's honest significant difference (HSD) test for multiple comparisons. The statistical significance threshold was taken as 0.05.

### Results

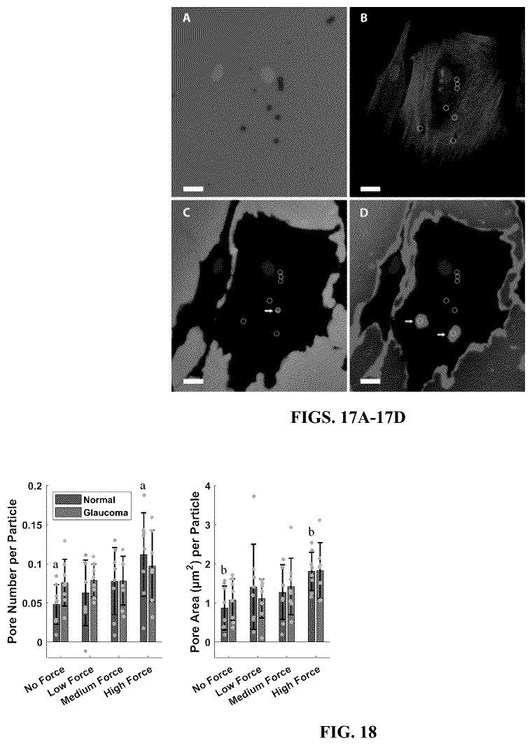

The fluorescent tracers reached and bound to the substrate at locations not covered by the cells, i.e., surrounding subconfluent cells but also at I-pore sites (FIG. 17-). Actin microstructure (FIG. 17B) was intact at the location of pores detected only by the first tracer (FIG. 17C) but was disrupted at some pore locations detected by the second tracer (FIG. 17D).

Even in the control wells, where no magnetic force was applied, some pores formed over the microbeads, indicating that the mere presence of the magnetic microbeads under the SC cells was sufficient to induce some I-pore formation (FIG. 18). Two-way ANOVA indicated that force significantly affected the number of pores (F (3,55)=3.62, p=0.019); however, the number of pores was not affected by the disease state of the cell strains (F (1,55)=0.61, p=0.439). There was no interaction effect between force and disease (F (3,55)=0.92, p=0.439). Similarly, when considering pore area, force was a significant factor (F (3,55)=3.95, p=0.013), whereas disease state was not (F (1,55)=0.01, p=0.906), and again, no interaction effect was observed (F (3,55)=0.43, p=0.733).

### DISCUSSION

The mechanobiological assay allows assessment of I-pores in SC cells using automated (masked) image processing. This study showed a correlation between focal magnetically-delivered force and pore formation, with more pores and greater pore area in normal SC cell strains in the high force condition vs. the no force condition. Some pores formed spontaneously (without magnets, i.e., Type I) through unclear mechanisms; likely due to intracellular tension within SC cells adhered to the underlying gelatin substrate which causes local stretch over microspheres sufficient to trigger pore formation in some cases.

In glaucomatous cells, I-pores were also detected, and pore number and area showed an increasing trend with applied force by ANOVA; however, pairwise differences between force levels did not reach statistical significance (post hoc Tukey's HSD test, p>0.05). Although based on only a single cell strain, this is consistently indicates that glaucomatous SC cells are less responsive to local force, perhaps due to their increased cell stiffness [10].

Disruption of actin microstructure only at some Type II pore locations indicates that I-pores form dynamically in the assay. All pores detected only with the first tracer and some pores detected by the second tracer were “closed” at the time of cell fixation, i.e. disruption of actin microstructure was not evident.

The following patents, applications and publications as listed below and throughout this document are hereby incorporated by reference in their entirety herein.

### REFERENCE LIST FOR EXAMPLE 1

1 Marchesi, V. & Gowans, J. L. The migration of lymphocytes through the endothelium of venules in lymph nodes: an electron microscope study. Proceedings of the Royal Society of London. Series B. Biological Sciences 159, 283-290 (1964).
2 Greenwood, J., Howes, R. & Lightman, S. The blood-retinal barrier in experimental autoimmune uveoretinitis. Leukocyte interactions and functional damage. Laboratory investigation; a journal of technical methods and pathology 70, 39-52 (1994).
3 Feng, D., Nagy, J. A., Dvorak, H. F. & Dvorak, A. M. Ultrastructural studies define soluble macromolecular, particulate, and cellular transendothelial cell pathways in venules, lymphatic vessels, and tumor-associated microvessels in man and animals. Microscopy research and technique 57, 289-326 (2002).
4 Lossinsky, A. S. & Shivers, R. Structural pathways for macromolecular and cellular transport across the blood-brain barrier during inflammatory conditions. Review. Histology and histopathology (2004).
5 Carman, C. V. et al. Transcellular diapedesis is initiated by invasive podosomes. Immunity 26, 784-797 (2007).
6 Neal, C. R. & Michel, C. C. Transcellular gaps in microvascular walls of frog and rat when permeability is increased by perfusion with the ionophore A23187. J Physiol 488 (Pt 2), 427-437, doi: 10.1113/jphysiol.1995.sp020977 (1995).
7 Neal, C. & Michel, C. Transcellular openings through frog microvascular endothelium. Experimental Physiology: Translation and Integration 82, 419-422 (1997).
8 Satchell, S. C. & Braet, F. Glomerular endothelial cell fenestrations: an integral component of the glomerular filtration barrier. American Journal of Physiology-Renal Physiology 296, F947-F956 (2009).
9 HOLMBERG, A. The fine structure of the inner wall of Schlemm's canal. AMA Archives of Ophthalmology 62, 956-958 (1959).
10 Tripathi, R. C. Ultrastructure of Schlemm's canal in relation to aqueous outflow. Experimental eye research 7, 335-IN336 (1968).
11 Tripathi, R. C. Mechanism of the aqueous outflow across the trabecular wall of Schlemm's canal. Experimental eye research 11, 116-IN139 (1971).
12 Ethier, C. R., Coloma, F. M., Sit, A. J. & Johnson, M. Two pore types in the inner-wall endothelium of Schlemm's canal. Investigative ophthalmology & visual science 39, 2041-2048 (1998).
13 Braakman, S. T. et al. Biomechanical strain as a trigger for pore formation in Schlemm's canal endothelial cells. Experimental eye research 127, 224-235 (2014).
14 Overby, D. R. et al. Altered mechanobiology of Schlemm's canal endothelial cells in glaucoma. Proc Natl Acad Sci USA 111, 13876-13881, doi: 10.1073/pnas. 1410602111 (2014).
15 Heijl, A. et al. Reduction of intraocular pressure and glaucoma progression: results from the Early Manifest Glaucoma Trial. Archives of ophthalmology 120, 1268-1279 (2002).
16 Tham, Y.-C. et al. Global prevalence of glaucoma and projections of glaucoma burden through 2040: a systematic review and meta-analysis. Ophthalmology 121, 2081-2090 (2014).
17 Varma, R., Lee, P. P., Goldberg, I. & Kotak, S. An assessment of the health and economic burdens of glaucoma. American journal of ophthalmology 152, 515-522 (2011).
18 Boland, M. V. et al. Comparative effectiveness of treatments for open-angle glaucoma: a systematic review for the US Preventive Services Task Force. Annals of internal medicine 158, 271-279 (2013).
19 Leske, M. C. et al. Factors for glaucoma progression and the effect of treatment: the early manifest glaucoma trial. Archives of ophthalmology 121, 48-56 (2003).
20 Lusthaus, J. & Goldberg, I. Current management of glaucoma. Medical Journal of Australia 210, 180-187 (2019).
21 Johnson, M. What controls aqueous humour outflow resistance? Experimental eye research 82, 545-557 (2006).
22 Ethier, C. R. The inner wall of Schlemm's canal. Experimental eye research 74, 161-172 (2002).
23 Johnson, M. et al. The pore density in the inner wall endothelium of Schlemm's canal of glaucomatous eyes. Investigative ophthalmology & visual science 43, 2950-2955 (2002).
24 Allingham, R. R. et al. The relationship between pore density and outflow facility in human eyes. Investigative ophthalmology & visual science 33, 1661-1669 (1992).
25 Stamer, W. D. & Acott, T. S. Current understanding of conventional outflow dysfunction in glaucoma. Current opinion in ophthalmology 23, 135 (2012).
26 Perkumas, K. & Stamer, W. Protein markers and differentiation in culture for Schlemm's canal endothelial cells. Experimental eye research 96, 82-87 (2012).
27 Stamer, W. D., Roberts, B. C., Howell, D. N. & Epstein, D. L. Isolation, culture, and characterization of endothelial cells from Schlemm's canal. Invest Ophthalmol Vis Sci 39, 1804-1812 (1998).
28 Pedrigi, R. M., Simon, D., Reed, A., Stamer, W. D. & Overby, D. R. A model of giant vacuole dynamics in human Schlemm's canal endothelial cells. Exp Eye Res 92, 57-66, doi: 10.1016/j.exer.2010.11.003 (2011).
29 Swain, D. L. et al. Schlemm's canal endothelium cellular connectivity in giant vacuole and pore formation in different flow-type areas: a serial block-face scanning electron microscopy study. Frontiers in Cell and Developmental Biology 10, 867376 (2022).
30 Cantu-Crouch, D., Howe, W. E. & McCartney, M. D. Comparison of SEM processing methods for cultured human lens epithelial cells grown on flat and microcarrier bead substrates. Microscopy research and technique 30, 419-426 (1995).
31 Braakman, S. T., Stamer, W. D. & Overby, D. R. A fluorescent permeability assay for Schlemm's canal endothelial cells in response to stretch. Investigative Ophthalmology & Visual Science 55, 5983-5983 (2014).
32 Siadat, S. M., Bertrand, J. A., Overby, D. R., Stamer, W. D. & Ethier, C. R. Development of a Schlemm's Canal “Inner Wall on a Chip” for High Content Screening. Investigative Ophthalmology & Visual Science 64, 50-50 (2023).
33 Vahabikashi, A. et al. Increased stiffness and flow resistance of the inner wall of Schlemm's canal in glaucomatous human eyes. Proc Natl Acad Sci USA 116, 26555-26563, doi: 10.1073/pnas. 1911837116 (2019).
34 Al-Humimat, G., Marashdeh, I., Daradkeh, D. & Kooner, K. Investigational Rho kinase inhibitors for the treatment of glaucoma. Journal of Experimental Pharmacology, 197-212 (2021).
35 Carman, C. V., Jun, C.-D., Salas, A. & Springer, T. A. Endothelial cells proactively form microvilli-like membrane projections upon intercellular adhesion molecule 1 engagement of leukocyte LFA-1. The Journal of Immunology 171, 6135-6144 (2003).
36 Sturdivant, J. M. et al. Discovery of the ROCK inhibitor netarsudil for the treatment of open-angle glaucoma. Bioorganic & medicinal chemistry letters 26, 2475-2480 (2016).
37 Vargas-Pinto, R., Gong, H., Vahabikashi, A. & Johnson, M. The effect of the endothelial cell cortex on atomic force microscopy measurements. Biophys J 105, 300-309, doi: 10.1016/j.bpj.2013.05.034 (2013).

### REFERENCE LIST FOR EXAMPLE 2

[1] Heijl, A. et al., Arch. Ophthalmol., 120.10:1268-1279, 2002.
[2] Weinreb, R., et al., The Lancet, 363.9422:1711-1720, 2004.
[3] Overby, D., et al., Exp. Eye Res., 88.4:656-670, 2009.
[4] Stamer, W. D., et al., IOVS, 52.13:9438-9444, 2011.
[5] Bill, A., et al., Acta Ophthalmologica, 50.3:295-320, 1972.
[6] Ethier, C. R., et al., IOVS, 39.11:2041-2048, 1998.
[7] Braakman, S., et al., Exp. Eye Res., 127:224-235, 2014.
[8] Ethier, C. R., Exp. Eye Res., 74.2:161-172, 2002.
[9] Braakman, S., (Thesis) Imperial College London, 2014.
[10] Overby, D., et al., PNAS, 111.38:13876-13881, 2014.

What is claimed is:

1. A biochip, comprising:
a substrate;
a mechanical stressor; and
a plurality of endothelial cells seeded on top of the mechanical stressor;
wherein the plurality of endothelial cells comprise a plurality of pores.

2. The biochip of claim 1, wherein the substrate comprises glass.

3. The biochip of claim 1, wherein the substrate further comprises a coating.

4. The biochip of claim 3, wherein the coating comprises biotinylated, crosslinked gelatin.

5. The biochip of claim 3, wherein the coating is configured to bind to a tracer at any regions of said coating not covered by the plurality of endothelial cells.

6. The biochip of claim 1, wherein the mechanical stressor comprises plurality of ferromagnetic particles.

7. The biochip of claim 1, wherein the plurality of endothelial cells comprise Schlemm's canal (SC) cells.

8. The biochip of claim 7, wherein the SC cells are healthy.

9. The biochip of claim 7, wherein the SC cells are glaucomatous.

10. The biochip of claim 1, wherein the plurality of pores comprise transcellular pores.

11. A method of making an endothelial barrier model, the method comprising:
a) providing a substrate comprising a mechanical stressor;
b) seeding a plurality of endothelial cells on top of the mechanical stressor; and
c) inducing transcellular pore formation in the plurality of endothelial cells using the mechanical stressor.

12. The method of claim 11, wherein the mechanical stressor comprises a plurality of ferromagnetic particles.

13. The method of claim 12, wherein step c) further comprises applying a magnetic force to the plurality of endothelial cells.

14. The method of claim 11, further comprising:
d) exposing the plurality of endothelial cells to a tracer, wherein the tracer binds to the substrate at any regions of said substrate not covered by the plurality of endothelial cells; and
e) detecting the tracer, thereby determining the presence of transcellular pores in the endothelial cells.

15. The method of claim 11, wherein the plurality of endothelial cells comprise Schlemm's canal (SC) cells.

16. The method of claim 15, wherein the SC cells are healthy.

17. The method of claim 15, wherein the SC cells are glaucomatous.

18. A method of screening agents that modulate transcellular pore formation, the method comprising:
i) providing an endothelial barrier model prepared by the method of claim 11;
ii) exposing the endothelial barrier model to a therapeutic agent; and
iii) determining the presence of transcellular pores in the plurality of endothelial cells as compared to an endothelial barrier model not exposed to the therapeutic agent.

19. The method of claim 18, wherein the therapeutic agent increases the formation of transcellular pores as compared to an endothelial barrier model not exposed to the therapeutic agent.

20. The method of claim 18, wherein step ii) occurs before, during, or after inducing transcellular pore formation in the plurality of endothelial cells using the mechanical stressor.
